(19) United States
(12) Patent Application Publication
(10) Pub. No.: US 2024/0219383 A1
(43) Pub. Date: Jul. 4, 2024

(54) DEVICE, SYSTEM, AND METHOD OF MEASURING CONCENTRATION OF ANALYTE IN BODILY FLUID SAMPLE

(71) Applicant: SPECLIPSE, INC., Seongnam-si (KR)

(72) Inventors: Sung Hyun PYUN, Seoul (KR); Wan Ki MIN, Seoul (KR); Chang Su JEON, Hwaseong-si (KR); In Su KIM, Seongnam-si (KR); Eun Yeong SONG, Seongnam-si (KR)

(73) Assignee: SPECLIPSE, INC., Seongnam-si (KR)

(21) Appl. No.: 18/113,108

(22) Filed: Feb. 23, 2023

(30) Foreign Application Priority Data
Dec. 28, 2022 (KR) 10-2022-0186865

(51) Int. Cl. G01N 33/543; G01N 33/58

(52) U.S. Cl. CPC G01N 33/54373; G01N 33/588; G01N 2470/06

(57) ABSTRACT

A method of measuring a concentration of an analyte in a bodily fluid sample is provided. The method includes introducing the bodily fluid sample containing the analyte into a test strip for a lateral flow assay (LFA) by a first volume to react the analyte with a metal nano probe for surface-enhanced Raman scattering (SERS), introducing a washing liquid into the test strip by a second volume after a first predetermined time interval has elapsed from the introduction of the bodily fluid sample, and performing a surface-enhanced Raman scattering (SERS)-based spectroscopic analysis on at least one of a test area and a control area of the test strip using a spectroscopic device.

### CROSS-REFERENCE TO RELATED APPLICATION

This application is based on and claims the benefit of priority to Korean Patent Application No. 10-2022-0186865, filed on Dec. 28, 2022, the disclosure of which is incorporated herein in its entirety by reference.

### BACKGROUND OF THE INVENTION

### Field of the Invention

The present disclosure relates generally to analysis of a bodily fluid sample, and more particularly, without limitation, to a device, a system, and a method of measuring a concentration of an analyte in a bodily fluid sample.

### Related Art

A method of measuring biometric information using a sample from a body is continuously developing. In the related art, an analysis method performed in laboratories, such as an enzyme-linked immunosorbent assay (ELISA), is mainly used for analysis of biometric information. However, as effectiveness of point of care (POC) is gradually improved and the need for rapid diagnosis increases, a scope of application for the POC is increasing.

Since the POC can perform on-site testing and provide the results in a short time, it can enable rapid response, and diagnostic methods are actively developed in a direction that can improve the accuracy of measurement results such as sensitivity, specificity, and reproducibility.

The point of care can be largely divided into a small-sized laboratory analysis device such as a Lab-On-a-Chip (LOC) and a disposable analysis device such as a lateral flow assay (LFA). The small-sized laboratory analysis device is a miniaturized analysis device for a clinical laboratory and can be used, for example, for gas analysis and electrolyte analysis in blood. Meanwhile, the small-sized laboratory analysis device has the advantage of being mobile, including a core technology of the laboratory analysis device, and being able to be used by even non-experts. However, the small-sized laboratory analysis device is relatively expensive because it is equipped with a micro sensor and MFC (Micro Fluidic Channel) on a chip with an area of several centimeters formed of glass, silicon, or plastic. The disposable analysis devices have been mainly used for albumin tests in urine or pregnancy tests in the related art, and have recently been widely used as simple diagnostic kits for infectious diseases caused by pandemics. The disposable analysis device is small and light, and has the advantage of being disposable in the form of a kit, so it does not require cleaning and is easy to use and diagnose. However, the disposable analysis device requires efforts to lower the unit cost of a disposable analyzer as much as possible in consideration of economic feasibility.

### SUMMARY OF THE INVENTION

A summary of specific embodiments disclosed in the present disclosure is presented below. It should be understood that aspects presented in the summary below are merely intended to provide a brief summary of specific embodiments and are not intended to limit the scope of the present disclosure. Therefore, it is noted in advance that the present disclosure may include various aspects not presented below.

The present disclosure and a concept of the present disclosure disclosed below provide a device, a system, and a method of measuring a concentration of an analyte in a bodily fluid sample.

In addition, the present disclosure and the concept of the present disclosure disclosed below provide a method of measuring a concentration of an analyte in a bodily fluid sample and a test strip used therefor.

In addition, the present disclosure and the concept of the present disclosure disclosed below provide a Raman spectroscopy method and a system therefor.

One task of the present disclosure is to provide a method and a system of measuring a concentration of an analyte in a bodily fluid sample by introducing the bodily fluid sample and a washing liquid into a test strip for lateral flow analysis and performing surface-enhanced Raman scattering-based spectroscopic analysis on the test area.

One task of the present disclosure is to provide a test strip for measuring a concentration of an analyte in a bodily fluid sample by introducing the bodily fluid sample and a washing liquid into the test strip to use a lateral flow assay.

One task of the present disclosure is to provide a Raman spectroscopic analysis method with respect to a test strip for measuring a concentration of an analyte in a bodily fluid sample.

However, problems to be solved by the present disclosure are not limited thereto, and may be expanded in various ways within a range that does not deviate from the spirit and area of the present disclosure.

Embodiments provide a device, a system, and a method of measuring a concentration of an analyte in a bodily fluid sample.

According to one embodiment, there is provided a method of measuring a concentration of an analyte in a bodily fluid sample, the method including: introducing the bodily fluid sample containing the analyte into a test strip for a lateral flow assay (LFA) by a first volume to react the analyte; introducing a washing liquid into the test strip by a second volume after a first predetermined time interval has elapsed from the introduction of the bodily fluid sample; and performing a surface-enhanced Raman scattering (SERS)-based spectroscopic analysis on at least one of a test area and a control area of the test strip using a spectroscopic device.

According to embodiments, a method of measuring a concentration of an analyte in a bodily fluid sample and a test strip used therefor are provided.

According to one embodiment, there is provided a test strip for measuring a concentration of an analyte in a bodily fluid sample using a lateral flow assay (LFA), the test strip including: a housing; a sample pad into which the bodily fluid sample containing the analyte and a washing fluid is introduced; a conjugate pad including a metal nano probe having a detection antibody binding to the analyte and a Raman indicator; a detection pad including a test area to which a capture antibody configured to capture a complex of the metal nano probe and the analyte is fixed and a control area configured to capture the metal nano probe; and an absorption pad disposed on a side surface opposite to a side surface of the conjugate pad of the detection pad to absorb the bodily fluid sample and the washing liquid and provide a transport force to the bodily fluid sample and the washing liquid.

According to embodiments, a Raman spectroscopy method and a system therefor are provided.

According to one embodiment, there is provided a Raman spectroscopic analysis method for a test strip for measuring a concentration of an analyte in a bodily fluid sample, the method including: preparing a test strip in which an analyte is fixed in a spectroscopic analysis target area; irradiating the spectroscopic analysis target area with laser using a spectroscopic device and measuring Raman scattering light from the spectroscopic analysis target area; and determining the concentration of the analyte based on a measured value of the Raman scattering light using an arithmetic device, in which the spectroscopic analysis target area includes a portion of at least one of a test area and a control area of the test strip.

The foregoing exemplary embodiments and other exemplary embodiments will be explained or made clear by the detailed description to be given later with respect to exemplary embodiments to be read in conjunction with the accompanying drawings.

The disclosed technology may have the following effects. However, it does not mean that a specific embodiment must include all of the following effects or only the following effects, so it should not be understood that the scope of rights of the disclosed technology is limited thereby.

According to one embodiment of the present disclosure, by sequentially injecting the bodily fluid sample and washing liquid into the test strip for the lateral flow analysis, the surface-enhanced Raman scattering-based spectroscopic analysis is performed on the test area to which the analyte is fixed, and thus, the concentration of the analyte in the bodily fluid sample can be measured. Therefore, the analytes remaining on a membrane can be more reliably moved to the analysis area and the accuracy of the measurement can be improved.

According to one embodiment of the present disclosure, the test strip into which the bodily fluid sample and washing liquid are introduced to measure the concentration of the analyte in the bodily fluid sample using the lateral flow assay (LFA) is provided. The test strip according to one aspect is provided with the absorption pad having an absorbent volume appropriate for the flow of the bodily fluid sample and the washing liquid, thereby enabling a washing procedure for the test strip for the lateral flow analysis.

According to one embodiment of the present disclosure, the Raman spectroscopic analysis method for the test strip for measuring the concentration of the analyte in the bodily fluid sample is provided. The spectroscopic analysis target area is set to exclude a side area where a large error occurs according to the flow of the bodily fluid sample and washing liquid, and thus, the spectroscopic analysis and the concentration measurement of the analytes can be performed with higher accuracy.

The above-described matters of the present disclosure are not intended to be an exhaustive list of all aspects of the present disclosure. It is to be understood that the present disclosure includes all methods, devices, and systems practicable from all suitable combinations of the various aspects set forth in the following detailed description and claims, as well as those summarized above.

### BRIEF DESCRIPTION OF THE DRAWINGS

FIG. 1 is a schematic flowchart of a method of measuring a concentration of an analyte in a bodily fluid sample according to one embodiment of the present disclosure.

FIG. 2 is a detailed flowchart of a step of introducing and reacting a bodily fluid sample in FIG. 1.

FIG. 3A is an example of preprocessing for the spectroscopic analysis step of FIG. 1.

FIG. 3B is a detailed flow chart of the spectroscopic analysis step in FIG. 1.

FIG. 4 is a block diagram illustrating a schematic configuration of a system of measuring the concentration of the analyte in the bodily fluid sample according to one embodiment of the present disclosure.

FIG. 5 is a perspective view of a test strip for measuring the concentration of the analyte in the bodily fluid sample using a lateral flow assay (LFA) according to one embodiment of the present disclosure.

FIG. 6 illustrates a pad configuration of the test strip of FIG. 5.

FIG. 7 illustrates introduction and action of washing liquid according to one aspect of the present disclosure.

FIG. 8 is an exemplary diagram of an opening arrangement of the test strip according to one aspect of the present disclosure.

FIG. 9 is a detailed exemplary diagram of a size of a second opening of FIG. 8.

FIG. 10 is an exemplary diagram of arrangement of a protective film on the test strip according to one aspect of the present disclosure.

FIG. 11 is an exemplary diagram of arrangement of a light blocking film of the test strip according to one aspect of the present disclosure.

FIG. 12 is an exemplary diagram of a trench for cutting an absorption pad of the test strip according to one aspect of the present disclosure.

FIG. 13 is an exemplary diagram of a cutting line for cutting the absorption pad of the test strip according to one aspect of the present disclosure.

FIG. 14 is an exemplary diagram of a cutting tool for cutting the absorption pad of the test strip according to one aspect of the present disclosure.

FIG. 15 is a side view of a pressurizing bulkhead for preventing backflow of a washing liquid in the test strip according to one aspect of the present disclosure.

FIG. 16 is a plan view of the pressurizing bulkhead for preventing the backflow of the washing liquid in the test strip according to one aspect of the present disclosure.

FIG. 17 is a plan view of a pressurizing protrusion for preventing the backflow of the washing liquid in the test strip according to one aspect of the present disclosure.

FIG. 18 illustrates an exemplary structure of a metal nano probe according to one aspect of the present disclosure.

FIG. 19 illustrates a hot spot formed on the metal nano probe of FIG. 18.

FIG. 20 illustrates an exemplary structure of a metal nano probe according to another aspect of the present disclosure.

FIG. 21 is a schematic flowchart of a Raman spectroscopic analysis method of the test strip for measuring the concentration of the analyte in the bodily fluid sample according to one embodiment of the present disclosure.

FIG. 22 is a detailed flowchart of a test strip preparation step of FIG. 21.

FIG. 23A is a detailed flowchart of a laser irradiation step and a Raman scattering light measurement step of FIG. 21.

FIG. 23B is a second detailed flowchart of the laser irradiation step and the Raman scattering light measurement step of FIG. 21.

FIG. 24A is a detailed flowchart of a measured value-based analyte concentration determination in FIG. 21.

FIG. 24B is a second detailed flow diagram of the measured value-based analyte concentration determination step in FIG. 21.

FIG. 25 illustrates a schematic configuration of a Raman spectroscopic analysis system according to one embodiment of the present disclosure.

FIG. 26 is an exemplary diagram of a target area for Raman spectroscopic analysis according to one aspect of the present disclosure.

FIG. 27 illustrates Raman spectroscopic analysis points and a moving path of a spectroscopic device according to one aspect of the present disclosure.

FIG. 28 is an exemplary diagram of determining a representative value according to a spectroscopic analysis measured value according to one aspect of the present disclosure.

FIG. 29 illustrates changes in the detection pad before and after washing at an antibody concentration of 3 mg/ml.

FIG. 30 illustrates changes in the detection pad before and after washing at an antibody concentration of 2 mg/ml.

FIG. 31 illustrates changes in the detection pad before and after washing at an antibody concentration of 1 mg/ml.

FIG. 32 illustrates response results of the test area at each concentration.

FIG. 33 illustrates results of Raman spectroscopic analysis at each concentration.

FIG. 34 illustrates a change of the Raman spectroscopic analysis measured value from one side surface of the test area to another side surface.

FIG. 35 illustrates the difference in Raman spectroscopic analysis results for the test area before and after drying.

FIG. 36 illustrates an analysis result of the Raman spectroscopic analysis result of FIG. 35.

FIG. 37 illustrates differences in Raman spectroscopic analysis results for the test area over time after washing.

FIG. 38 illustrates an analysis result of the Raman spectroscopic analysis result of FIG. 37.

### DETAILED DESCRIPTION OF THE INVENTION

Since the present disclosure can include various changes and have various embodiments, specific embodiments are illustrated in the drawings and described in detail.

However, this is not intended to limit the present disclosure to specific embodiments, and should be understood to include all modifications, equivalents, and substitutes included in the spirit and scope of the present disclosure.

Terms such as first and second may be used to describe various components, but the components should not be limited by the terms. These terms are only used for the purpose of distinguishing one component from another. For example, a first component may be termed a second component, and similarly, a second component may be termed a first component, without departing from the scope of the present disclosure. The terms and/or include any combination of a plurality of related recited items or any of a plurality of related recited items.

It is understood that when a component is referred to as being “coupled” or “connected” to another component, it may be directly connected or connected to another element, but other components may exist in the middle. Meanwhile, when a component is referred to as “directly coupled” or “directly connected” to another component, it should be understood that no other element exists in the middle.

Terms used in the present disclosure are only used to describe specific embodiments, and are not intended to limit the present disclosure. Singular expressions include plural expressions unless the context clearly dictates otherwise. In the present disclosure, the terms “include” or “have” are intended to designate that there is a feature, number, step, operation, component, part, or combination thereof described in the specification, but it should be understood that the presence or addition of one or more other features, numbers, steps, operations, components, parts, or combinations thereof is not precluded.

Unless defined otherwise, all terms used herein, including technical or scientific terms, have the same meaning as commonly understood by one of ordinary skill in the art to which the present disclosure belongs. Terms such as those defined in commonly used dictionaries should be interpreted as having a meaning consistent with the meaning in the context of the related art, and unless explicitly defined in the present application, they should not be interpreted in an ideal or excessively formal meaning.

Hereinafter, with reference to the accompanying drawings, preferred embodiments of the present disclosure will be described in more detail. In order to facilitate overall understanding in the description of the present disclosure, the same reference numerals are used for the same components in the drawings, and redundant descriptions of the same components are omitted.

Since the embodiments described in the present disclosure are intended to clearly explain the spirit of the present disclosure to those skilled in the art to which the present disclosure belongs, the present disclosure is not limited by the embodiments described in the present disclosure, and the scope of the present invention should be construed to include modifications or variations that do not depart from the spirit of the present invention.

The terminology used in the present disclosure is a selection of general terms that are currently widely used as much as possible in the technical field to which the present disclosure belongs, but this may have different meanings depending on the intentions of those skilled in the art, customs, or the emergence of new technologies in the technical field to which the present invention belongs. However, in the case where a specific term is defined and used with an arbitrary meaning, the meaning of the term will be separately described. Therefore, the terminology used in the present disclosure should be interpreted based on the actual meaning of the term and the general contents of the present disclosure, not the simple name of the term.

The drawings attached to the present disclosure are for easily explaining the present disclosure, the shapes illustrated in the drawings may be exaggerated or abbreviated as necessary to help the understanding of the present disclosure, and thus, the present disclosure is not limited by the drawings.

In the present disclosure, when it is determined that a detailed description of a known configuration or function related to the present disclosure may obscure the gist of the present disclosure, a detailed description thereof will be omitted if necessary.

According to one aspect of the present disclosure, there is provided a method of measuring a concentration of an analyte in a bodily fluid sample, the method may include: introducing the bodily fluid sample containing the analyte into a test strip for a lateral flow assay (LFA) by a first volume to react the analyte with a metal nano probe for surface-enhanced Raman scattering (SERS); introducing a washing liquid into the test strip by a second volume after a first predetermined time interval has elapsed from the introduction of the bodily fluid sample; and performing a surface-enhanced Raman scattering (SERS)-based spectroscopic analysis on at least one of a test area and a control area of the test strip using a spectroscopic device.

According to one aspect, the introducing of the bodily fluid sample by the first volume to react may include loading the bodily fluid sample by a sample pad of the test strip, binding the metal nano probe including a Raman indicator and a detection antibody included in a conjugate pad of the test strip and the analyte included in the bodily fluid sample, transporting the bodily fluid sample by an absorption pad provided on a side surface opposite to a side surface of the conjugate pad of the detection pad of the test strip, capturing a complex of the metal nano probe and the analyte by a capture antibody fixed to the test area of the detection pad, and capturing the metal nano probe by the control area of the detection pad.

According to one aspect, the introducing of the washing liquid by the second volume includes transporting the washing liquid loaded on the sample pad via the conjugate pad and the detection pad by the absorption pad.

According to one aspect, the method may further, after the introducing of the washing liquid, transporting complexes located at an area prior to the test area of the detection pad or the conjugate pad among complexes of the metal nano probe and the analyte to the test area by the washing liquid.

According to one aspect, the method may further include, after the introducing of the washing liquid, removing non-specific binding by substances other than the analyte to the capture antibody of the test area by the washing liquid.

According to one aspect, the second volume of the washing liquid may be set to be equal to or greater than a first threshold volume which is a volume allowing complexes located at an area prior to the test area of the detection pad or the conjugate pad among complexes of the metal nano probe and the analyte to be transported to the test area.

According to one aspect, the second volume of the washing liquid may be set to be smaller than a second threshold volume which is a volume allowing the complex of the metal nano probe and the analyte captured by the capture antibody of the test area to be lost.

According to one aspect, at least one of the first threshold volume and the second threshold volume of the washing liquid may be determined based on at least one of a first volume of the bodily fluid sample, a viscosity of the bodily fluid sample, a viscosity of the washing liquid, a type of the analyte, a type of the metal nano probe, and an absorbent volume of the absorbent pad.

According to one aspect, the absorption pad may have an absorbent volume equal to or greater than a sum of the first volume and the second volume.

According to one aspect, the method may further include cutting at least a portion of the absorption pad of the test strip after a second predetermined time interval has elapsed from the introduction of the washing liquid, in which the SERS-based spectroscopic analysis may be performed after the cutting of a portion of the absorption pad.

According to one aspect, the method may further include drying the test area for a predetermined third time interval after the washing liquid is introduced, in which the SERS-based spectroscopic analysis may be performed after the drying of the test area.

According to one aspect, the metal nano probe may include a metal nano core formed of a first metal, a Raman indicator layer surrounding the metal nano core and having one or more Raman indicators, a metal shell surrounding the Raman indicator layer and formed of a second metal, and a detection antibody fixed to the metal shell and binding to the analyte.

According to one aspect, the metal nano core and the metal shell may be spaced apart from each other by the at least one Raman indicator to form a hot spot for generating the surface-enhanced Raman scattering in the Raman indicator layer.

According to one aspect, the metal nano core may be formed of gold (Au), and the metal shell may be formed of silver (Ag).

According to one aspect, the metal nano probe may further include at least one additional Raman indicator disposed on an outer surface of the metal shell.

According to one aspect, the Raman indicator and the additional Raman indicator may be different from each other.

According to one aspect, one of the Raman indicator and the additional Raman indicator may be MBA, and the other of the Raman indicator and the additional Raman indicator may be MGITC.

According to one aspect, the performing of the SERS-based spectroscopic analysis may include determining a first measured value of the test area based on spectroscopic analysis, determining a second measured value of the control area based on spectral analysis, determining a dryness evaluation value of the test area based on at least one of the first measured value and the second measured value, and determining the concentration of the analyte according to the dryness evaluation value and the first measured value.

According to another aspect of the present disclosure, there is provided a system of measuring a concentration of an analyte in a bodily fluid sample, the system including: A spectroscopic device and a test strip for a lateral flow assay, in which the test strip receives the bodily fluid sample including an analyte by a first volume, reacts the analyte with a metal nano probe for surface-enhanced Raman scattering, and receives a washing liquid by a second volume after a first predetermined time interval from the introduction of the bodily fluid sample, and the spectroscopic device performs a surface-enhanced Raman scattering-based spectroscopic analysis on at least one of a test area and a control area of the test strip.

According to one aspect of the present disclosure, there is provided a test strip for measuring a concentration of an analyte in a bodily fluid sample using a lateral flow assay (LFA), the test strip including: a housing; a sample pad into which the bodily fluid sample containing the analyte and a washing fluid is introduced; a conjugate pad including a metal nano probe having a detection antibody binding to the analyte and a Raman indicator; a detection pad including a test area to which a capture antibody configured to capture a complex of the metal nano probe and the analyte is fixed and a control area configured to capture the metal nano probe; and an absorption pad disposed on a side surface opposite to a side surface of the conjugate pad of the detection pad to absorb the bodily fluid sample and the washing liquid and provide a transport force to the bodily fluid sample and the washing liquid.

According to one aspect, the washing liquid may be introduced into the sample pad after the first predetermined time interval has elapsed from the introduction of the bodily fluid sample.

According to one aspect, the absorption pad may have an absorbent volume equal to or greater than a sum of a first volume, which is an input amount of the bodily fluid sample, and a second volume, which is an input amount of the washing liquid.

According to one aspect, the housing may include a first opening disposed above the absorption pad; and a second opening exposing at least a portion of the test area and the control area to the outside.

According to one aspect, the second opening may be configured to have a width that is smaller than a width of the test area by a first predetermined length or more from both side surfaces.

According to one aspect, the first length may be determined such that a side surface area of the test area in a width direction is covered by the housing, and the side surface area in the width direction may be an area in which the density of the metal nano probe remaining after transport of the bodily fluid sample and the washing liquid differs from the density of the metal nano probe in the central area in the width direction by a predetermined critical error or more.

According to one aspect, the second opening may be configured to have a width capable of securing a predetermined number or more of Raman spectroscopic analysis points.

According to one aspect, the test strip may further include a protective film made of a light-transmitting material and covering the second opening, in which the protective film may have a transparency of at least a first transparency higher than a transparency for light rays having wavelengths other than the first wavelength for light rays of a first wavelength for the Raman spectroscopic analysis of the test area.

According to one aspect, the protective film may be configured to have a transparency equal to or less than a second predetermined transparency with respect to light rays having wavelengths other than the first wavelength.

According to one aspect, the housing may include a first opening disposed above the absorption pad, a test area opening exposing at least a portion of the test area to the outside; and a control area opening exposing at least a portion of the control area to the outside.

According to one aspect, the test strip may further include a light blocking film having a transparency equal to or less than a predetermined third transparency and covering the opening of the test area.

According to one aspect, the light blocking film may be configured to be removable, discoloration of the control area may be exposed based on the control area opening, and the light blocking film may be removed prior to Raman spectroscopic analysis.

According to one aspect, the test strip may further include a cutting unit for cutting out at least a portion of the absorption pad after absorption of the washing liquid by the absorption pad.

According to one aspect, the cutting unit may include at least one of a trench formed in the detection pad or the absorption pad in a width direction, a cutting line including a cutting portion and a coupling portion alternately formed in the width direction of the detection pad or the absorption pad, and a cutting tool coupled to an operation unit outside the housing and configured to cut the detection pad or the absorption pad by being controlled by the operation unit.

According to one aspect, the test strip may further include a blocking unit fixed after absorption of the washing liquid by the absorption pad to prevent the washing liquid from flowing backward from the absorption pad to the detection pad.

According to one aspect, the blocking unit may include at least one of a pressurizing bulkhead formed in a width direction of the detection pad or the absorption pad and pressurizing the detection pad or the absorption pad, and a plurality of pressurizing protrusions disposed on at least a partial area of the detection pad or the absorption pad to press the detection pad or the absorption pad.

According to one aspect, by the transport of the washing liquid, among complexes of the metal nano probe and the analyte, complexes located in an area prior to the test area of the detection pad or the conjugate pad may be transported to the test area.

According to one aspect, non-specific binding by a substance other than the analyte to the capture antibody of the test area may be removed by transport of the washing liquid.

According to one aspect, the metal nano probe may include a metal nano core formed of a first metal; a Raman indicator layer surrounding the metal nano core and having one or more Raman indicators, a metal shell surrounding the Raman indicator layer and formed of a second metal, and a detection antibody fixed to the metal shell and binding to the analyte.

According to one aspect, the metal nano core and the metal shell may be spaced apart from each other by the at least one Raman indicator to form a hot spot for generating surface-enhanced Raman scattering in the Raman indicator layer.

According to one aspect of the present disclosure, there is provided a Raman spectroscopic analysis method for a test strip for measuring a concentration of an analyte in a bodily fluid sample, the method including: preparing a test strip in which an analyte is fixed in a spectroscopic analysis target area; irradiating the spectroscopic analysis target area with laser using a spectroscopic device and measuring Raman scattering light from the spectroscopic analysis target area; and determining the concentration of the analyte based on a measured value of the Raman scattering light using an arithmetic device, in which the spectroscopic analysis target area includes a portion of at least one of a test area and a control area of the test strip.

According to one aspect, the spectroscopic analysis target area may be configured to have a width that is smaller than a width of the test area by a first predetermined length or more from both side surfaces.

According to one aspect, the first length may be determined to exclude a side surface area in the width direction of the test area from the spectroscopic analysis target area, and the side surface area in the width direction may be an area in which the density of the fixed analyte differs by a predetermined threshold error or more from the density of the analyte in the central area in the width direction.

According to one aspect, the spectroscopic analysis target area may be configured to have a width capable of securing a predetermined number or more of Raman spectroscopic analysis points.

According to one aspect, the preparing of the test strip may include introducing a bodily fluid sample containing an analyte into a test strip for a lateral flow assay (LFA) to react, and introducing a washing liquid into the test strip after a predetermined first time interval has elapsed from the introduction of the body fluid sample.

According to one aspect, the preparing of the test strip may further include drying the test area for a predetermined third time interval after the washing liquid is introduced.

According to one aspect, the preparing of the test strip may further include removing a light blocking film attached to an opening corresponding to a test area of a test strip housing.

According to one aspect, the measuring of the Raman scattering light may be performed before a predetermined threshold time interval has elapsed from the completion of preparation of the test strip.

According to one aspect, in the measuring of the Raman scattering light, irradiation may be performed with a laser beam having a beam spot size exceeding a first size generating combustion of the analyte-bound metal nano probe.

According to one aspect, in the measuring of the Raman scattering light, irradiation may be performed with a laser having a beam spot size of a second size or less capable of securing a predetermined number or more of Raman spectroscopic analysis points within the spectroscopic analysis target area.

According to one aspect, in the measuring of the Raman scattering light, scattered light of a plurality of Raman spectroscopic analysis points within the analysis target area may be measured using a stage device configured to horizontally move the spectroscopic device in any one of a longitudinal direction and a width direction in the analysis target area within the analysis target area.

According to one aspect, the spectroscopic device may be configured to stop laser irradiation while moving from a first spectroscopic analysis point to a second spectroscopic analysis point among the plurality of Raman spectroscopic analysis points.

According to one aspect, the measuring of the Raman scattering light may include setting a moving path of the spectroscopic device for the plurality of Raman spectroscopic analysis points, measuring Raman scattering light for at least some of odd-numbered Raman spectroscopic analysis points within the moving path, and measuring Raman scattering light for at least some of even-numbered Raman spectroscopic analysis points within the moving path.

According to one aspect, the determining of the concentration of the analyte may include determining a representative value of a measured value for each column of columns of Raman spectroscopic analysis points arranged in a longitudinal direction of the spectroscopic analysis target area, and determining an overall representative value for the spectroscopic analysis target area based on the representative value for each column.

According to one aspect, the measuring of the Raman scattering light may include determining a first measured value for the test area and determining a second measured value for the control area, and in the determining of the concentration of the analyte, the concentration of the analyte may be determined based on the first measured value and the second measured value.

According to one aspect, the determining of the concentration of the analyte may include determining a dryness evaluation value for the test area based on at least one of the first measured value and the second measured value and determining the concentration of the analyte according to the dryness evaluation value and the first measured value.

According to another aspect of the present disclosure, there is provided a system for Raman spectroscopic analysis, the system including: a test strip in which an analyte is fixed to a spectroscopic analysis target area to measure a concentration of an analyte in a bodily fluid sample; a spectroscopic device irradiating to the spectroscopic analysis target area with a laser and measuring Raman scattering light from the spectroscopic analysis target area; a stage device configured to horizontally move the spectroscopic device in any one of a longitudinal direction and a width direction within the analysis target area; and an arithmetic device determining the concentration of the analyte based on a measured value of the Raman scattering light, in which the spectroscopic analysis target area may include a portion of at least one of a test area and a control area of the test strip.

### Outline

As described above, a scope of application of point of care is gradually increasing, and lateral flow analysis is most widely used.

The lateral flow analysis (LFA) method, which is a representative disposable analysis device, is an analysis technology that can detect an analyte in a sample through immunoassay technology using nanoparticles and sample flow using a membrane. A typical LFA test strip includes a rectangular support made of a plastic material, and is configured to include a sample pad, a conjugate pad, a detection pad, and an absorption pad sequentially disposed on the support from one side to the other side. LFA is widely used for on-site analysis because it is inexpensive, easy to carry and quickly detect, and can be easily used by the general public without professional skills.

In LFA, the analyte contained in the sample introduced into the sample pad flows along the membrane (detection pad) by capillary action while binding to the metal nanoparticles-detection antibody complex immobilized on the conjugate pad. At this time, metal nanoparticles which are detection indicators bind to the capture antibody fixed to the test area, color development is performed based on the plasmonic effect, and thus, the result can be confirmed.

Basic LFA evaluates the detection factor with the naked eye by the color development of metal nanoparticles that form a complex with an analyte. Therefore, there is a problem in that analytical sensitivity is not excellent and quantitative analysis is difficult. However, in recent years, the need for measuring the concentration of the analyte is increasing rather than simply detecting the presence or absence of the analyte, and thus, research on high-sensitivity LFA with a low level of detection (LOD) is also being actively conducted.

In this regard, a surface-enhanced Raman scattering (SERS)-based detection method is an analysis method that can overcome a detection sensitivity limit of Raman spectroscopy. This analysis method is a method that can quantify a target substance by measuring a change in intensity of the amplified characteristic SERS peak of the Raman indicator (Raman reporter molecule).

When the Raman indicator is adsorbed on a rough metal surface and exposed to laser light, a hot junction is generated to generate electromagnetic and/or chemical enhancement of the Raman indicator, thereby greatly increasing the SERS signal. Therefore, it is expected to solve the problem of low sensitivity, which is a disadvantage of the conventional Raman detection method.

According to the method of measuring the concentration of the analyte in the bodily fluid sample according to one aspect of the present disclosure, the analyte concentration in the bodily fluid sample can be measured by performing surface-enhanced Raman scattering-based spectroscopic analysis on the test area and/or control area of the test strip for the lateral flow analysis into which the bodily fluid sample is introduced. Therefore, it is possible to detect even a small amount of analyte, thereby improving a level of detection (LOD) of the test strip. Moreover, it is possible to perform quantitative analysis on the degree to which an analyte exists as well as a simple diagnosis of whether an analyte is present in the body fluid sample.

According to one aspect of the present disclosure, a predetermined period of time has elapsed after the bodily fluid sample is introduced into the test strip for the lateral flow analysis, then the washing liquid is introduced. Accordingly, the washing liquid can be absorbed into the absorption pad through the membrane of the test strip. Therefore, the effect of removing non-specific binding present in the test area can also be expected while allowing the complex of the analyte and metal nano probe remaining on the membrane to reach the test area.

In addition, according to one aspect of the present disclosure, the test strip is provided for measuring the concentration of the analyte in the bodily fluid sample using the lateral flow assay (LFA) in which a bodily fluid sample and washing liquid are introduced. For the application of the washing liquid to the lateral flow analysis, various structural features are disclosed, including, for example, an absorbent volume of an appropriate absorption pad, a means for cutting off the absorption pad, or a means for preventing backflow of the washing liquid. Therefore, the test strip having characteristics optimized for the application of washing liquid is provided.

In addition, the design of the opening for the test area of the test strip housing is disclosed in order to apply the surface-enhanced Raman scattering-based spectroscopic analysis for the lateral flow analysis. Accordingly, the laser for spectroscopic analysis can efficiently reach the analysis target area while preventing an effect of deterioration of detection intensity due to the spectroscopic analysis due to light exposure prior to the spectroscopic analysis.

According to the Raman spectroscopic analysis method according to one aspect of the present disclosure, the spectroscopic analysis is efficiently performed on the spectroscopic analysis target area of the test strip for measuring the concentration of the analyte in the bodily fluid sample, and the concentration of the analyte can be determined based on this. According to one aspect, a preliminary procedure for setting the analysis target area and the spectroscopic analysis may be performed in consideration of the application of the washing liquid. Therefore, it is possible to sufficiently secure the detection intensity according to the spectroscopic analysis, while also improving the consistency of the experimental results.

Hereinafter, a device, a system, and a method of measuring a concentration of an analyte in a bodily fluid sample according to embodiments of the present disclosure, a method of measuring a concentration of an analyte in a bodily fluid sample, a test strip used therefor, a Raman spectroscopic analysis method, and the system therefore will be explained in detail.

### Terms

Specific technical terms may be used in the present disclosure, and terms used in the present disclosure will be described below in order to establish definitive support for terms used in the present disclosure.

The following is a summary of preferred definitions of some terms used in the present disclosure. The following definitions are only exemplary definitions and are not exhaustive or limiting.

In the present disclosure, a “bodily fluid sample” includes, but is not limited to, samples such as tissue, cells, whole blood, serum, plasma, saliva, sputum, cerebrospinal fluid, or urine containing an analyte to be analyzed.

In the present disclosure, a term “analyte” is a substance to be detected present in a sample, and the type of analyte is not limited as a substance commonly used in the art, but for example, may be peptide nucleic acid (PNA), locked nucleic acid (LNA), peptide, polypeptide, protein, RNA, or DNA.

In the present disclosure, a term “probe” means a substance capable of specifically binding to an analyte to be detected in a sample, and means a substance that can specifically perform quantitative analysis and/or presence of the analyte in a sample through the above binding.

In the present disclosure, a term “test area” may refer to an area in which an analyte-probe complex generated by specific binding of the analyte to be detected and the probe is captured, and for example, may be a test line, but not limited thereto.

In the present disclosure, a term “control area” is an area for verifying whether the test strip is normally operating, and may mean an area in which metal nanoparticles can be captured, and may be, for example, a control line, but is not limited thereto.

In the present disclosure, a term “longitudinal direction” refers to a direction in which the bodily fluid sample and/or the washing liquid is transported in the test strip.

In the present disclosure, a term “width direction” may mean a direction perpendicular to a length direction of the test strip, the detection pad, the test area, the control area, or the housing.

### Method and System of Measuring Concentration of Analyte

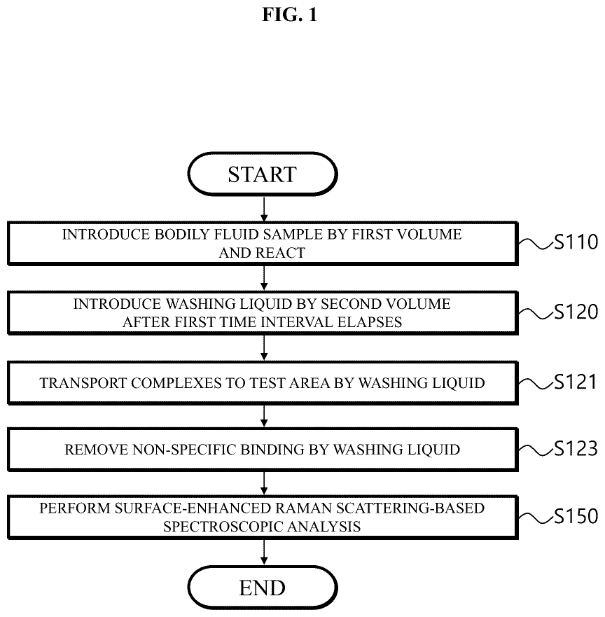

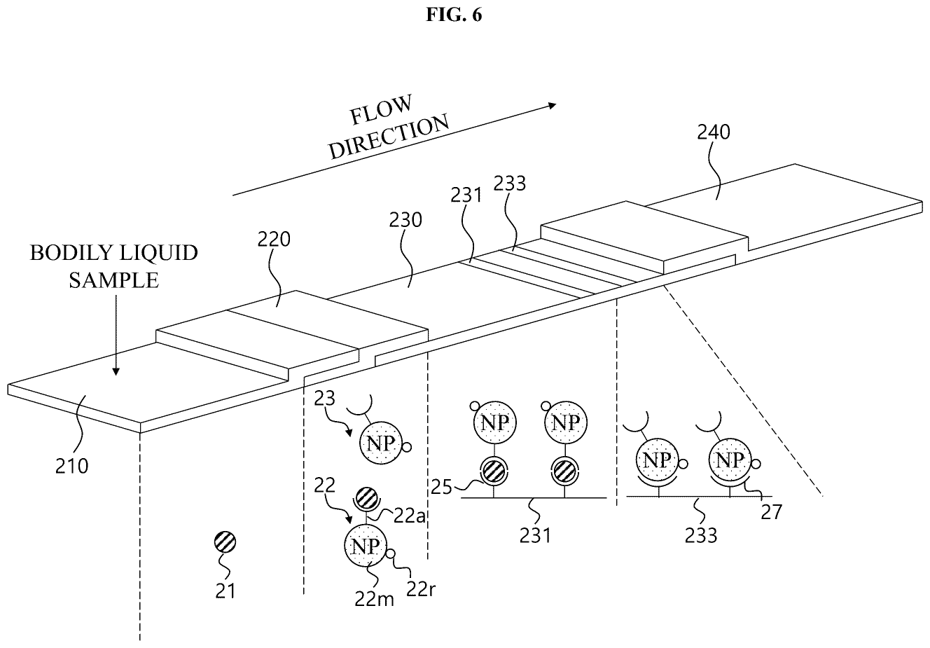

FIG. 1 is a schematic flowchart of a method of measuring a concentration of an analyte in a bodily fluid sample according to one embodiment of the present disclosure. FIG. 6 illustrates a pad configuration of the test strip according to one aspect. Hereinafter, a method of measuring the concentration of the analyte in the bodily fluid sample according to one embodiment of the present disclosure will be described with reference to FIGS. 1 and 6.

As illustrated in FIG. 1, the method of measuring the concentration of the analyte according to one embodiment of the present disclosure may include a step (S110) of introducing the bodily fluid sample into the test strip to react the analyte with a metal nano probe for surface-enhanced Raman scattering (SERS), a step (S120) of introducing a washing liquid into the test strip after a predetermined period of time has elapsed, and a step (S150) of performing surface-enhanced Raman scattering-based spectroscopic analysis.

More specifically, according to the method of measuring the concentration of the analyte according to one embodiment of the present disclosure, first, the bodily fluid sample containing the analyte may be introduced into the test strip for a lateral flow assay (LFA) by a first volume, and the analyte may be reacted with the metal nano probe for the surface-enhanced Raman scattering (SERS) (S110). The pad structure of one exemplary test strip is illustrated in FIG. 6 and is described in detail again later in the present disclosure.

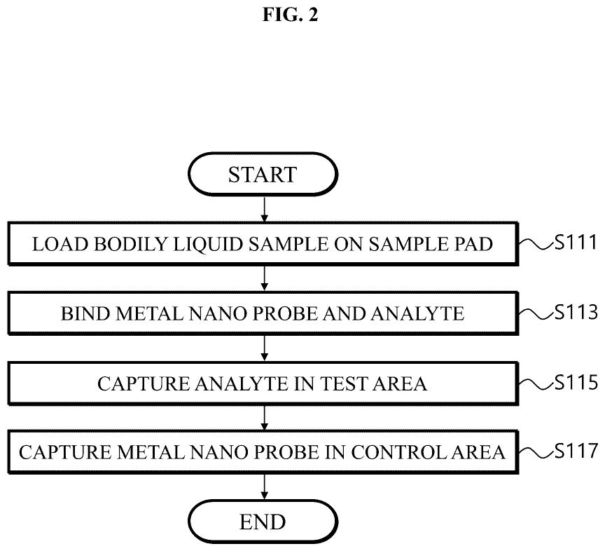

FIG. 2 is a detailed flowchart of the step of introducing and reacting the bodily fluid sample in FIG. 1. As illustrated in FIGS. 2 and 6, according to one embodiment of the present disclosure, first, the bodily fluid sample is loaded into a sample pad 210 of a test strip 200 (S111). The bodily fluid sample may be introduced by a first predetermined volume. Here, the first volume may be determined differently according to a type of a body fluid or an analyte to be analyzed. As illustrated in FIG. 6, a bodily fluid sample may include an analyte 21 to be subjected to detection and concentration analysis according to the present disclosure.

Due to the capillarity caused by a porous membrane-based detection pad 230 and/or absorption capacity of an absorption pad 240, the bodily fluid sample introduced into the sample pad 210 moves along a flow direction as illustrated in FIG. 6. Therefore, the bodily fluid sample passes through a conjugate pad 220, and the analyte 21 included in the bodily fluid sample binds to a metal nano probe 22 included in the conjugate pad 220 of the test strip (S113).

More specifically, the metal nano probe 22 may include a Raman indicator 22r and a detection antibody 22a, and the Raman indicator 22r and the detection antibody 22a may be configured to be fixed to metal nanoparticles 22m. Here, the Raman indicator 22r may be a “Raman reporter molecule” and may be selected from among substances generating Raman scattering. When the metal nanoparticles 22m are fixed in the test area, the metal nanoparticles 22m may be generated based on the plasmonic effect between the metal nanoparticles and act to visually indicate whether or not they are detected, and may provide a hot spot for the Raman indicator 22r to generate the surface-enhanced Raman scattering. The detection antibody 22a may be an antibody that specifically binds to the analyte according to one embodiment of the present disclosure. The metal nano probe 22 may bind to the analyte 21 by having the detection antibody 22a to form the metal nano probe-analyte complex. The formed metal nano probe-analyte complex moves along the flow direction illustrated in FIG. 6.

Among the metal nano probes included in the conjugate pad 220, at least some of the metal nano probes 23 may not bind to the analyte 21, and the metal nano probes 23 that do not bind to the analyte are also illustrated in FIG. moves along the flow direction.

More specifically, at least due to the absorption capacity of the absorption pad 240 provided on a side surface opposite to a side surface of the conjugate pad 220 of the detection pad 230 of the test strip, the bodily fluid sample and the analyte contained therein and the metal nano probe-analyte complex or the metal nano probe not binding to the analyte are moved.

Therefore, a capture antibody 25 fixed to the test area 231 of the detection pad 230 may capture the complex of the metal nano probe and the analyte (S115). The capture antibody 25 may be configured to perform specific binding with the analyte 21, and thus the metal nano probe-analyte complex including the analyte 21 may be fixed to the test area 231. Here, the capture antibody 25-analyte 21-metal nano probe 22 can be understood as forming a sandwich complex. The detection antibody 22a may be bound to a primary side of the analyte 21, and the capture antibody 25 may bind to a secondary side of the analyte 21, but is not limited thereto. Regardless of the names of the detection antibody and the capture antibody, it should be understood that any antibody that bind to the analyte 21 may be used.

Subsequently, the control area 233 of the detection pad 230 may capture the metal nano probe (S117). The capture antibody 27 fixed to the control area 233 may capture at least one of the metal nano probe-analyte complex and/or the metal nano probe not bound to the analyte. FIG. 6 illustrates that the capture antibody 27 binds to metal nanoparticles, for example, but this is merely exemplary and various types of antibodies for capturing metal nano probes may be applied.

Through the above steps, the input and reaction of the bodily fluid sample to the test strip 200 (S110) may be completed.

Referring back to FIG. 1, after a first predetermined time interval elapses from the introduction of the bodily fluid sample, the washing liquid may be introduced into the test strip by a second volume (S120). More specifically, when the washing liquid is introduced into the sample pad 210 by the second volume, the washing liquid loaded on the sample pad 210 may be transported to the absorption pad 240 through the conjugate pad 220 and the detection pad 230 by at least one of the absorption capacity of the absorption pad 240 and/or the capillary phenomenon of the membrane constituting the detection pad 230.

In laboratory-performed analytical techniques such as an enzyme-linked immunosorbent assay (ELISA), a procedure for performing washing after the formation of a complex for an analyte by an antibody is generally included, but a procedure of additionally administering the washing liquid after the introduction of the sample to be analyzed in the strip for lateral flow analysis has not been attempted. According to one embodiment of the present disclosure, the bodily fluid sample is introduced into the test strip for the lateral flow analysis, and after a predetermined time has elapsed, the washing liquid is additionally introduced by the second volume, so that the washing liquid can be transported to the absorption pad 240 through the conjugate pad 220 and the detection pad 230.

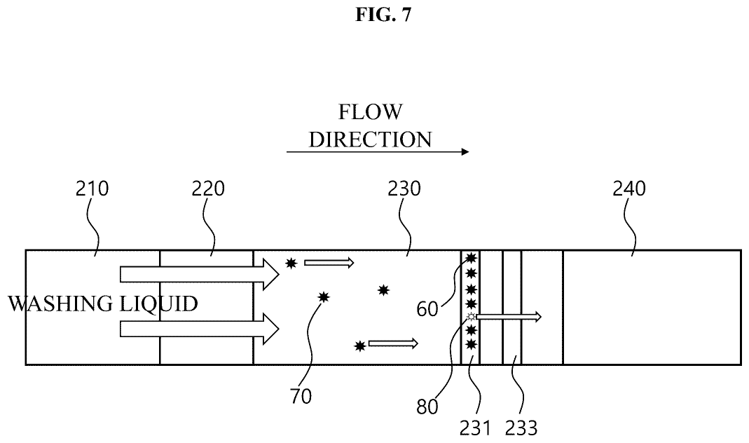

FIG. 7 illustrates the introduction and action of the washing liquid according to one aspect of the present disclosure. As illustrated in FIG. 7, in the test area 231 of the test strip 200 where the bodily fluid sample is introduced and the reaction is completed, the reaction substance and the complexes 60 of the metal nano probe may be captured and fixed by the capture antibody in the test area. However, when only the bodily fluid sample is introduced, even after the reaction and transport of the bodily fluid sample and the analyte contained therein are completed, some of the complexes of the analyte and metal nano probe formed in the conjugate pad 220 may not reach the test area 231 and may remain in various parts of the membrane. In addition, it is preferable for more accurate analysis results that only the analyte-deceleration nano probe complexes captured by the capture antibody are fixed to the test area 231, but a substance 80 other than the analyte may be present. For example, the substance 80 other than the analyte may form non-specific binding to the test area 231 and/or the capture antibody.

According to one embodiment of the present disclosure, the aforementioned problem can be solved by introducing the washing liquid (S120) after a predetermined time elapses after the introduction of the bodily fluid sample.

For example, according to one aspect, the method according to one embodiment of the present disclosure may further include, after the introduction of the washing liquid, a step (S121) of transporting complexes 70 located at an area prior to the test area of the conjugate pad or detection pad among complexes of the metal nano probe and the analyte to the test area 231 by the washing liquid.

In addition, according to one aspect of the present disclosure, the method according to one embodiment of the present disclosure may further include, after the introduction of the washing liquid, a step (S123) of removing the non-specific binding caused by the substance 80 other than the analyte for the capture antibody of the test area 231 by the washing liquid.

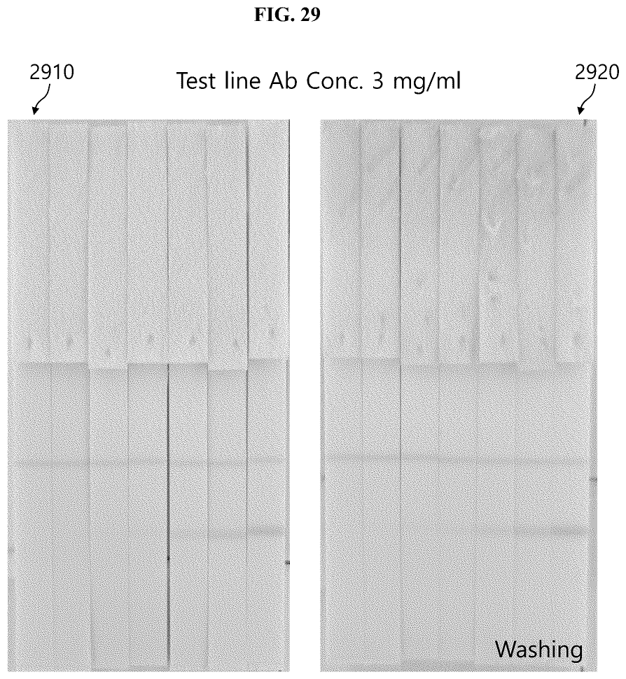

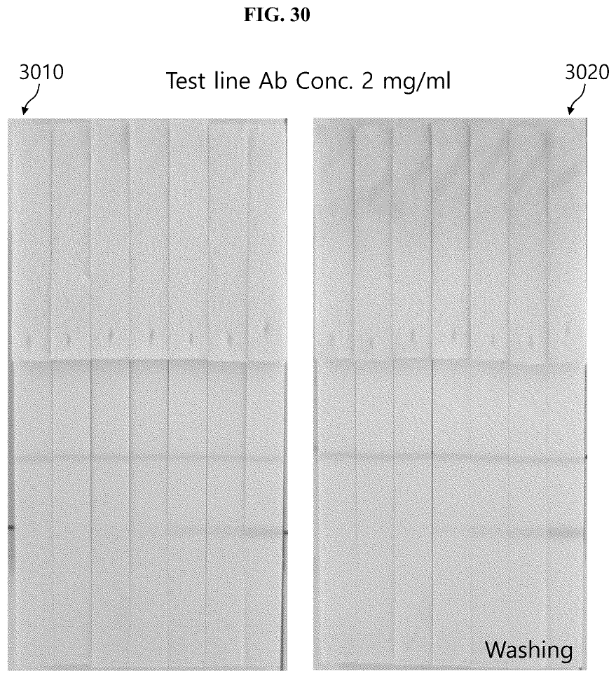

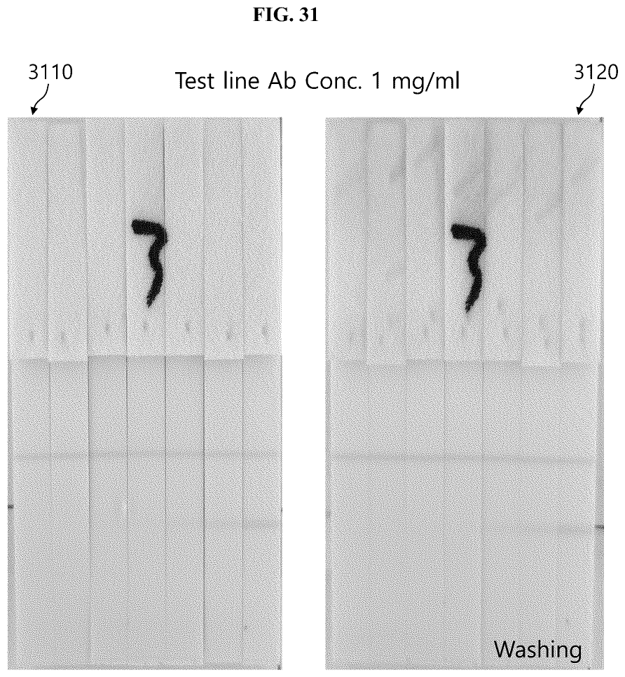

In this regard, FIG. 29 illustrates changes in the detection pad before and after the washing at an antibody concentration of 3 mg/ml, FIG. 30 illustrates changes in the detection pad before and after washing at an antibody concentration of 2 mg/ml, and FIG. 31 illustrates changes in the detection pad before and after washing at an antibody concentration of 1 mg/ml.

As illustrated in FIG. 29, at the antibody concentration of 3 mg/ml, compared with the detection line of the detection pad before performing the washing 2910 and/or parts other than the control line, after performing the washing 2920, it can be seen that a red tone disappears and becomes closer to white in parts other than the detection line and/or control line of the detection pad. This may indicate that some metal nano probes existing in the area before the detection line of the detection pad are moved by the washing liquid and the amount of remaining metal nano probes is reduced. In addition, when the detection line before performing the washing 2910 and the detection line after performing the washing 2920 are compared with each other, it can be confirmed that the degree of color development of the detection line is slightly improved by performing the washing. This is because the complex of the analyte-metal nano probe remaining on the detection pad before the occurrence area is further transported and captured by the capture antibody of the detection line, and the non-specific binding existing in the detection line is removed, so that substances other than the analyte are removed from the detection line.

As illustrated in FIG. 30, at the antibody concentration of 2 mg/ml, compared with the detection line of the detection pad before performing the washing 3010 and/or parts other than the control line, after performing the washing 3020, it can be seen that the red tone disappears and becomes closer to white in parts other than the detection line and/or the control line of the detection pad. This may indicate that some of the metal nano probes existing in the area before the detection line of the detection pad are moved by the washing liquid and the amount of remaining metal nano probes is reduced. In addition, when the detection line before performing the washing 3010 and the detection line after performing the washing 3020 are compared with each other, it can be confirmed that the degree of color development of the detection line is somewhat improved by the washing. This is because the complex of the analyte-metal nano probe remaining on the detection pad before the generated area is further transported and captured by the capture antibody of the detection line, and the non-specific binding existing in the detection line is removed, so that substances other than the analyte are removed from this detection line.

As illustrated in FIG. 31, at an antibody concentration of 1 mg/ml, when the detection line of the detection pad before performing the washing 3110 and/or parts other than the control line are compared with each other, after performing the washing 3020, it can be seen that the red tone disappears and becomes closer to white in parts other than the detection line and/or the control line of the detection pad. This may indicate that some of the metal nano probes existing in the area before the detection line of the detection pad are moved by the washing liquid and the amount of remaining metal nano probes is reduced. In addition, when the detection line before performing the washing 3110 and the detection line after performing the washing 3120 are compared with each other, it can be confirmed that the degree of color development of the detection line is somewhat improved by the washing. This is because the complex of the analyte-metal nano probe remaining on the detection pad before the generated area is further transported and captured by the capture antibody of the detection line, and the non-specific binding existing in the detection line is removed, so that substances other than the analyte are removed from this detection line.

As a result, a difference in degree occurs depending on the difference in the antibody concentration, but when comparing before and after washing, it was confirmed that the residues of the metal nano probe existing in the previous area of the detection line of the detection pad were removed, and the color development of the detection line was also improved.

In order to express the effect of such washing, it may be required to introduce the washing liquid after an appropriate time elapses from the time when the bodily fluid sample is introduced into the test strip. According to one aspect of the present disclosure, washing liquid may be introduced into the test strip after a lapse of a first predetermined time interval from the introduction of the bodily fluid sample. Here, the first time interval may be set to be longer than a time required for the bodily fluid sample and the analyte contained therein to react with the metal nano-probe in the test strip and move to the absorption pad, and to be shorter than a time when the bodily fluid sample is transported to the absorption pad and the moisture remaining on the conjugate pad or membrane is excessively dried and the residues are fixed.

Meanwhile, the volume of washing liquid introduced into the sample pad 210 may be considered. According to one aspect of the present disclosure, the washing liquid may be added as much as the second volume.

Here, according to one aspect, the second volume of the washing liquid may be set to be equal to or greater than a first threshold volume which is a volume allowing complexes located at the area prior to the test area of the detection pad or the conjugate pad among complexes of the metal nano probe and the analyte to be transported to the test area. That is, the volume of the washing liquid sufficient to sufficiently move the residue remaining on the membrane should be introduced at least.

Meanwhile, according to one aspect, the second volume of the washing liquid may be set to be smaller than a second threshold volume which is a volume allowing the complex of the metal nano probe and the analyte captured by the capture antibody of the test area to be lost. That is, when an excessive amount of washing liquid is introduced, a problem may occur that the complex of the analyte-metal nano probe, which is properly captured by the capture antibody in the test area and fixed to the test area, is rather separated. According to one aspect of the present disclosure, it is possible to introduce the washing liquid having a volume smaller than the volume at which the specific binding formed in the test area is lost.

Meanwhile, the threshold for the volume of the washing liquid may be changed by various factors. For example, even in the case of the same volume, the intensity of flow may be different for each washing liquid having a different viscosity. Therefore, according to one aspect of the present disclosure, at least one of the first threshold volume and the second threshold volume of the washing liquid may be determined based on at least one of a first volume of the bodily fluid sample, a viscosity of the bodily fluid sample, a viscosity of the washing liquid, a type of the analyte, a type of the metal nano probe, and an absorbent volume of the absorbent pad. In this regard, an appropriate volume of washing liquid may be different depending on how much the bodily fluid sample is loaded and what the viscosity of the bodily fluid sample is. In addition, according to the viscosity of the washing liquid, the larger the viscosity of the washing liquid, the larger the volume of the washing liquid to be introduced can be set. Since the transport force required may vary depending on the type of analyte and/or metal nano probe, for example, a lot of washing liquid may be introduced for an analyte and/or metal nano probe having a large mass.

Meanwhile, according to one aspect of the present disclosure, since the washing liquid is added to the test strip 200 in addition to the bodily fluid sample, the absorbent volume of the absorption pad 240 may be determined by reflecting this. According to one aspect, the absorption pad 240 may be configured to have an absorbent volume equal to or greater the sum of a first volume, which is an input amount of the bodily fluid sample, and a second volume, which is an input amount of the washing liquid.

Referring back to FIG. 1, after introducing the washing liquid, the surface-enhanced Raman scattering-based spectroscopic analysis may be performed (S150). However, according to one aspect of the present disclosure, preprocessing of the test strip 200 may be performed prior to spectroscopic analysis.

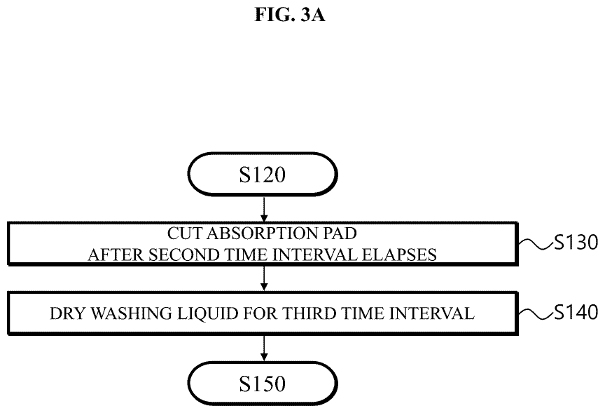

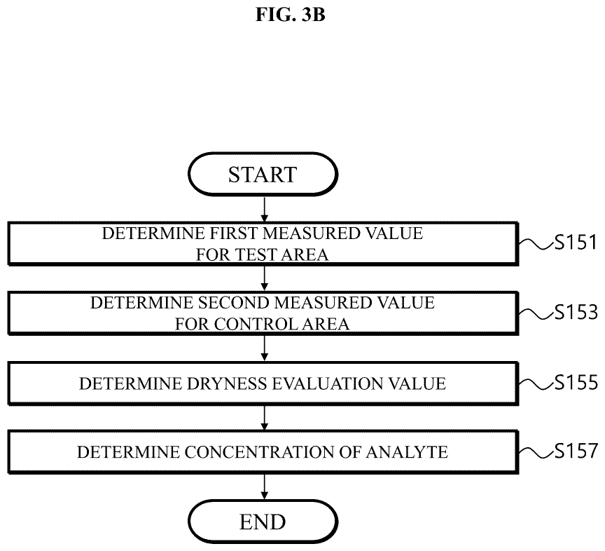

FIG. 3A is an example of preprocessing for the spectroscopic analysis step of FIG. 1. As illustrated in FIG. 3A, according to one aspect of the present disclosure, prior to the surface-enhanced Raman scattering-based on spectroscopic analysis, after a second predetermined time interval elapses from the introduction of the washing liquid, at least a portion of the absorption pad of the test strip may be cut (S130). When the washing liquid is introduced into the strip, the washing liquid is absorbed by the absorption pad 240 through the sample pad 210, conjugate pad 220, and detection pad 230. Here, since the amount of liquid that can be absorbed by the absorption pad 240 is greater than that of the detection pad 230, the washing liquid remaining in the absorption pad 240 can be dried faster than the washing liquid absorbed in the absorption pad 240 after a certain period of time elapses after the washing liquid is introduced. In this case, the washing liquid absorbed by the absorption pad 240 may flow back to the detection pad 230 again. In order to prevent this, after the second time interval, which is the time interval sufficient for the introduced washing liquid to be absorbed by the absorption pad 240, according to one aspect of the present disclosure, at least a portion of the absorption pad is cut to prevent a backflow from the absorption pad 240 toward the detection pad 23. To this end, a cutting unit for cutting the absorption pad 240 may be provided in the test strip 200 according to one aspect of the present disclosure, which will be described in detail later in the present disclosure.

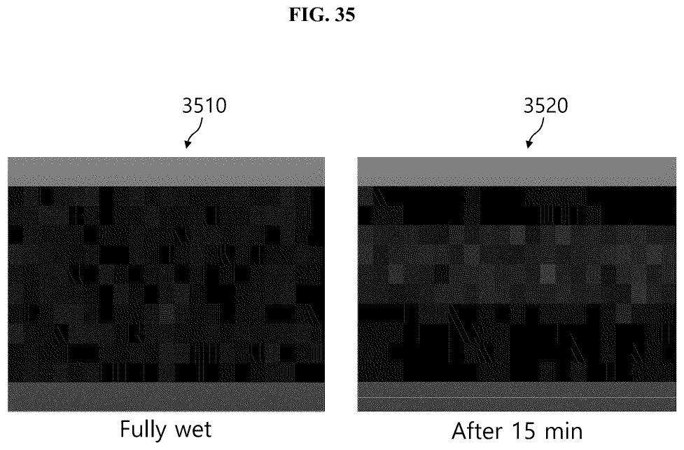

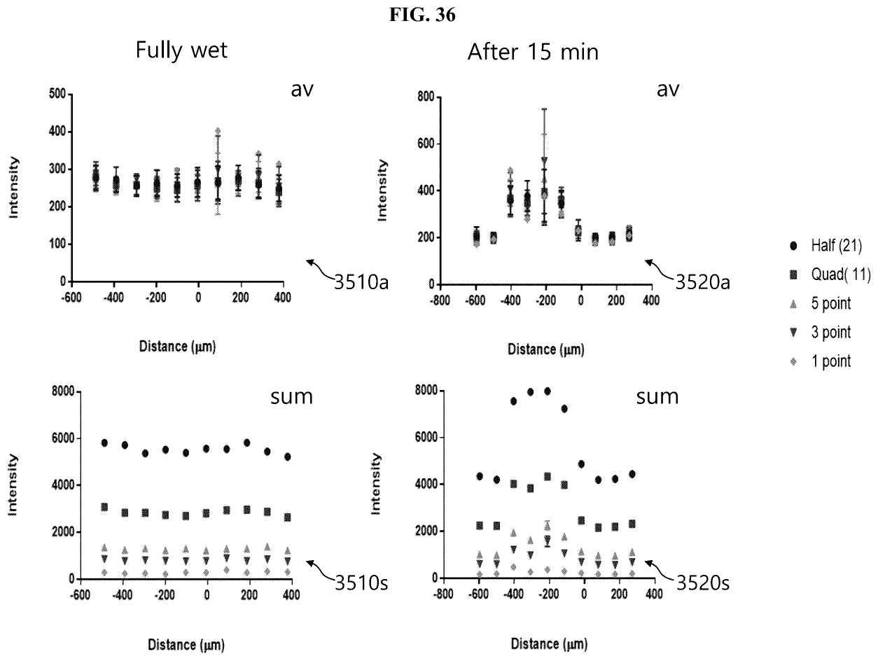

Referring back to FIG. 3A, according to one aspect of the present disclosure, the test area may be dried for a predetermined third time interval after the washing liquid is introduced (S140). As will be described later with reference to FIGS. 35 to 36 in the present disclosure, when sufficient drying is not achieved for the bodily fluid sample and/or washing liquid present in the test area 231 of the test strip 200, the intensity according to spectroscopic analysis may decrease. Therefore, according to one aspect of the present disclosure, the test area is sufficiently dried for the predetermined third time interval after the washing liquid is introduced, thereby preventing a decrease in the intensity in a spectroscopic analysis observation value.

Referring back to FIG. 1, the surface-enhanced Raman scattering (SERS)-based spectroscopic analysis may be performed on at least one of the test area or control area of the test strip using the spectroscopic device (S150).

In this regard, FIG. 3B is a detailed flow diagram of the spectroscopic analysis step of FIG. 1 according to one aspect of the present disclosure. According to one aspect of the present disclosure, the step (S150) of performing SERS-based spectroscopic analysis may include a step (S151) of determining a first measured value of the test area based on the spectroscopic analysis, a step (S153) of determining a second measured value of the control area based on the spectroscopic analysis, a step (S155) of determining a dryness evaluation value of the test area based on at least one of the first measured value and the second measured value, and a step (S157) of determining the concentration of the analyte according to the dryness evaluation value and the first measured value.

In other words, according to one aspect of the present disclosure, in determining the concentration of the analyte, not only the spectroscopic analysis result of the test area but also the spectroscopic analysis result of the control area may be further considered. As described above, when the test area and/or the control area are wet by the bodily fluid sample and/or the washing liquid, the intensity of the signal according to spectroscopic analysis may vary accordingly. Therefore, a dryness evaluation value indicating how dry the test area may be determined by comparing the first measured value of the test area according to the spectroscopic analysis and the second measured value of the control area according to the spectroscopic analysis with each other, and the concentration of the analyte according to the first measured value may be determined by reflecting the dryness evaluation value. For example, when the dryness degree is determined as the second value indicating less dryness than the first value, for the signal intensity for the same test area, a higher concentration may be determined when the drying degree is the second value than when the first value.

In addition, a surface-enhanced Raman scattering spectroscopic analysis procedure according to one aspect of the present disclosure will be described later in the present disclosure.

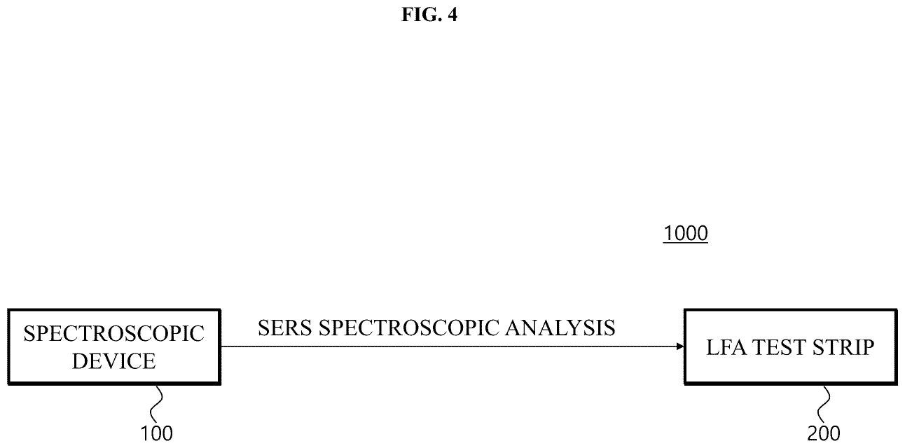

FIG. 4 is a block diagram illustrating a schematic configuration of a system for measuring the concentration of the analyte in the bodily fluid sample according to one embodiment of the present disclosure. As illustrated in FIG. 4, a system 1000 for measuring the concentration of the analyte in a bodily fluid sample according to one aspect of the present disclosure may include a spectroscopic device 100 and a lateral flow assay (LFA) test strip 200.

The test strip 200 is configured to introduce the bodily fluid sample containing the analyte by the first volume to react, and to introduce the washing liquid by the second volume after the first predetermined time interval has elapsed from the introduction of the bodily fluid sample.

The spectroscopic device 100 may be configured to perform the surface-enhanced Raman scattering (SERS)-based spectroscopic analysis on at least one of the test area and the control area of the test strip.

The method of measuring the concentration of the analyte in the bodily fluid sample according to one aspect of the present disclosure described above may be performed using the system 1000 illustrated in FIG. 4 and at least one of the components.

### Test Strip

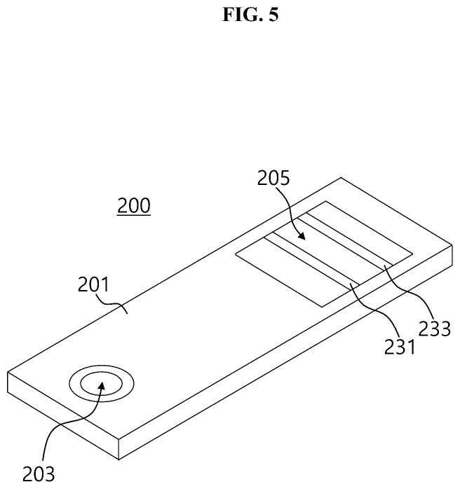

FIG. 5 is a perspective view of the test strip for measuring the concentration of the analyte in the bodily fluid sample using the lateral flow assay (LFA) according to one embodiment of the present disclosure. As illustrated in FIG. 5, the test strip 200 according to one aspect of the present disclosure may include a housing 201. The housing 201 may include a first opening 203 for introducing the bodily fluid sample and/or the washing liquid and a second opening 205 for exposing the test area 231 and/or control area 233 to the outside of the housing 201 so that the areas 231 and 233 can be visually checked.

FIG. 6 illustrates a pad configuration of the test strip of FIG. 5. As illustrated in FIGS. 5 and 6, the test strip 200 for lateral flow analysis according to one aspect of the present disclosure may include the sample pad 210, the conjugate pad 220, the detection pad 230, and the absorption pad 240 inside the housing 201.

As discussed above, the bodily fluid sample containing the analyte and the washing liquid are introduced into the sample pad 210. The conjugate pad 220 includes the detection antibody that binds to an analyte and the metal nano probe having the Raman indicator. The detection pad 230 includes the test area 231 to which the capture antibody configured to capture a complex of the metal nano probe and the analyte is fixed and the control area 233 configured to capture the metal nano probe. The absorption pad 240 is disposed on the side surface opposite to the side surface of the conjugate pad of the detection pad 230 to absorb bodily fluid sample and washing liquid and provides a transport force to the bodily fluid sample and washing liquid.

As described above in relation to the method of measuring the concentration of the analyte in the bodily fluid sample according to one aspect of the present disclosure, in the sample pad 210, the washing liquid may be introduced after the first predetermined time interval has elapsed from the introduction of the bodily fluid sample, the metal nano probes remaining prior to the test area 231 of the detection pad 230 may be additionally transported, and the non-specific binding existing in the test area 231 may be removed.

Moreover, according to one aspect, the absorbent volume of the absorption pad 240 may be determined in consideration of the volumes of both the bodily fluid sample and the washing liquid. For example, the absorption pad 240 may have an absorbent volume equal to or greater than the sum of a first volume which is an input amount of the bodily fluid sample to the test strip, and a second volume which is an input amount of washing liquid.

Hereinafter, the structure of the test strip 200 according to aspects of the present disclosure will be described in more detail.

### Opening

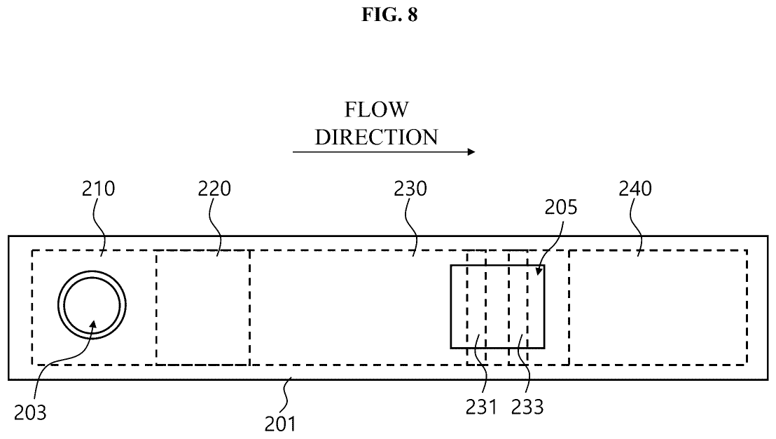

FIG. 8 is an exemplary diagram of an opening arrangement of a test strip according to one aspect of the present disclosure. As illustrated in FIG. 8, according to one aspect of the present disclosure, the housing 201 of the test strip 200 may include a first opening 203 disposed above the sample pad 210, and a second opening 205 that exposes at least a portion of the test area 231 and the control area 233 to the outside. As illustrated in FIG. 8, the remaining parts except for the first opening 203 and the second opening 205 may be protected by the housing 201.

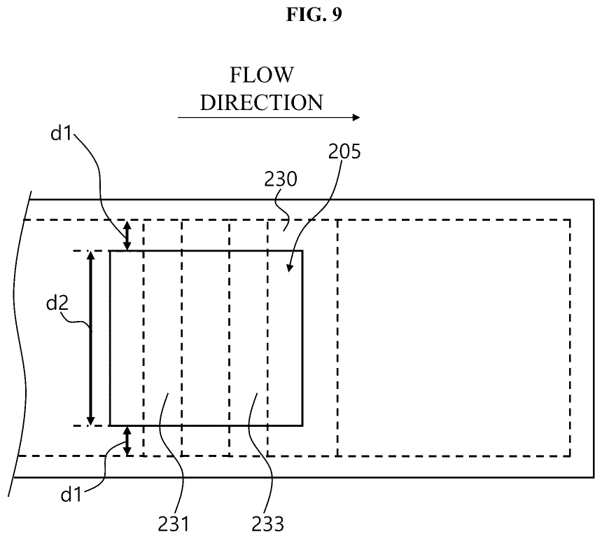

FIG. 9 is a detailed illustration of a size of the second opening of FIG. 8. In the test strip 200 according to one aspect of the present disclosure, the surface-enhanced Raman scattering-based spectroscopic analysis may be performed on at least one of the test area 231 and/or the control area 233 to determine the concentration of the analyte. Here, the area to be subjected to spectroscopic analysis may be set to at least a part of at least one of the test area 231 and/or the control area 233 to improve accuracy of the analysis. To this end, the second opening 205 according to one aspect of the present disclosure may be formed in a size such that at least a portion of the test area 231 and/or the control area 233 is exposed to the outside of the housing.

According to one aspect, the second opening 205 may be configured to have a width reduced from both sides by a first predetermined length d1 or more than the width of the test area 231. More specifically, according to one aspect, the first length d1 may be determined such that a side area of the test area 231 in the width direction is covered by the housing 201.

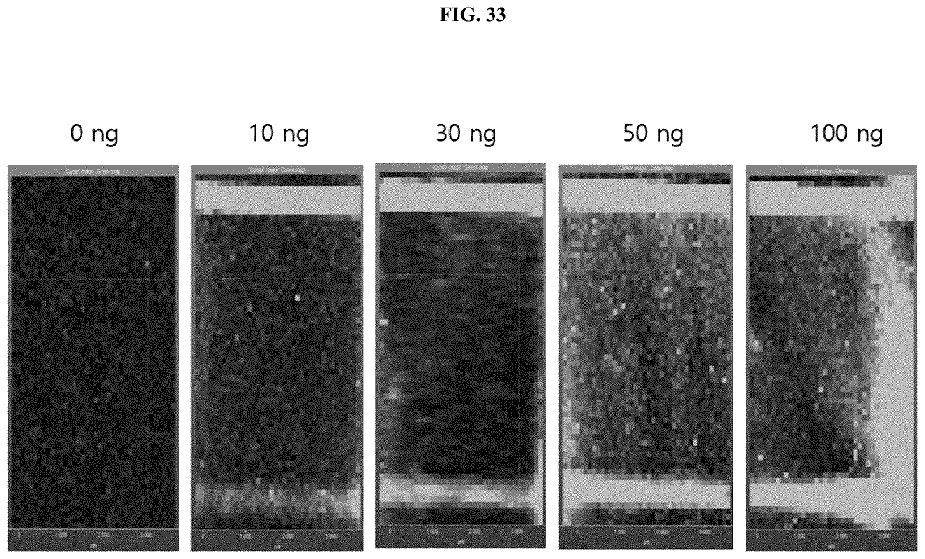

The side area in the width direction of the test area 231 covered by the housing 201 may be an area in which a density of the metal nano probe remaining after the transport of the bodily fluid sample and the washing liquid is different from the density of the metal nano probe in a central area (width d2) in the width direction by a predetermined threshold error or more. In this regard, referring to FIG. 33, Raman spectroscopic analysis results of the test area at various concentrations are illustrated. As illustrated in FIG. 33, it can be seen that excessively strong Raman scattering light is detected in both side areas of the test area in the width direction. This characteristic is due to the nature of a lateral flow assay (LFA), as the bodily fluid sample and/or washing liquid transport along the flow direction while carrying the nano-metal probe including the Raman indicator. Accordingly, it is assumed that more nanometallic probes are captured or retained in both side areas of the flow direction. According to one aspect of the present disclosure, side areas in the width direction in which an excessively high intensity of Raman scattering light is detected compared to the center area in the width direction may be excluded from the target area of Raman spectroscopic analysis. To this end, according to one side, the width of the second opening 205 formed in the housing 201 may be reduced from the width of the test area 231 by the first length d1 on both sides to have the second length d2. Therefore, in the analysis of Raman scattering light, the side area that is too different from other areas in the width direction may be excluded from the target of the spectroscopic analysis.

However, according to one aspect, the second opening 205 formed in the housing 201 may be configured to have a width capable of securing a predetermined number or more of Raman spectroscopic analysis points. For example, as illustrated in FIG. 35, according to one aspect of the present disclosure, the spectroscopic analysis of the test area 231 is performed on a plurality of Raman spectroscopic analysis points in the width direction and the length direction. The size of the point may vary according to the setting, but in order to secure a Raman spectroscopic analysis mapping result with a desired resolution, the desired number of spectroscopic analysis points according to the set size should be included in the width direction. When the width of the second opening 205 is too small, only a limited number of spectroscopic analysis points can be arranged in the width direction. Therefore, the width of the second opening 205 may not be reduced as compared with the length at which the desired number of spectroscopic analysis points can be arranged.

### Protective Film and Light Blocking Film

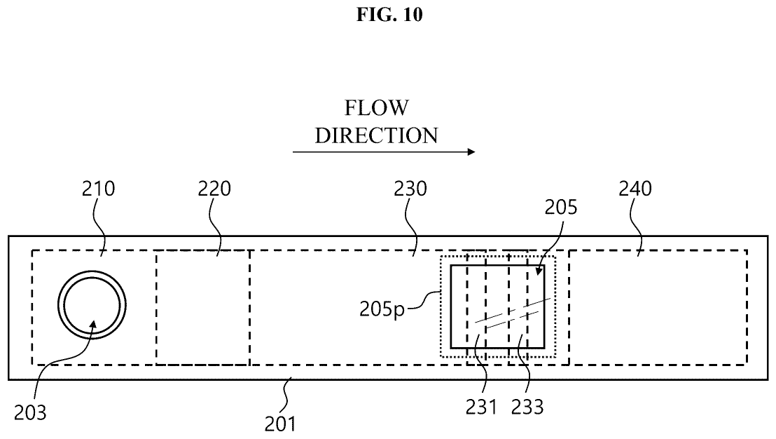

FIG. 10 is an exemplary diagram of arrangement of a protective film on the test strip according to one aspect of the present disclosure. As illustrated in FIG. 10, according to one aspect, the housing may include a protective film 205p covering the second opening 205. Unlike the first opening 203 into which a bodily fluid sample and/or washing liquid is introduced, a separate liquid is not introduced into the second opening 205. Accordingly, so as illustrated in FIG. 10, the protective film 205p formed of a light-transmitting material and covering the second opening 205 is provided to secure a view of the test area 231 and/or control area 233 while preventing physical exposure to the outside.

Meanwhile, according to one aspect of the present disclosure, since the Raman spectroscopic analysis is performed on the test area 231 and/or the control area 233, for the light ray of the first wavelength for the Raman spectroscopic analysis of the test area, the protective film 205p may have a transparency equal to or more than a first transparency that is higher than the transparency for light rays having wavelengths other than the first wavelength. That is, at least in the wavelength band of the laser for performing the Raman spectroscopic analysis, the protective film 205p has the transparency equal to or more than the first transparency so that the laser can be well delivered from the external spectroscopic device to the test area 231 and/or control area 233 which are the targets of the spectroscopic analysis, and the intensity reduction of the laser does not go beyond the acceptable level.

According to one aspect, the protective film may be configured to have a transparency less than the first transparency for a light ray having a different wavelength from the laser for the Raman spectroscopic analysis, and the protective film may have lower transparency for light rays of other wavelengths. Accordingly, the effect of light exposure on the Raman indicators existing in the test area 231 and/or the control area 233 can be minimized. That is, according to one aspect, the protective film 205p may be configured to have the transparency equal to or less than a second predetermined transparency with respect to light rays having wavelengths other than the first wavelength.

The Raman spectroscopic analysis may be performed by radiating the Raman indicator equipped in the metal nano probe with laser and measuring intensity of the Raman scattering light from the Raman indicator. However, it was found that such a decrease in intensity may occur when the Raman indicator is exposed to light for a long time. According to the embodiments of the present disclosure, in order to solve the problem of the decrease in intensity in performing the Raman spectroscopic analysis, it may be configured to minimize light exposure to the Raman indicator for displaying the concentration of the analyte which is the target of the spectroscopic analysis.

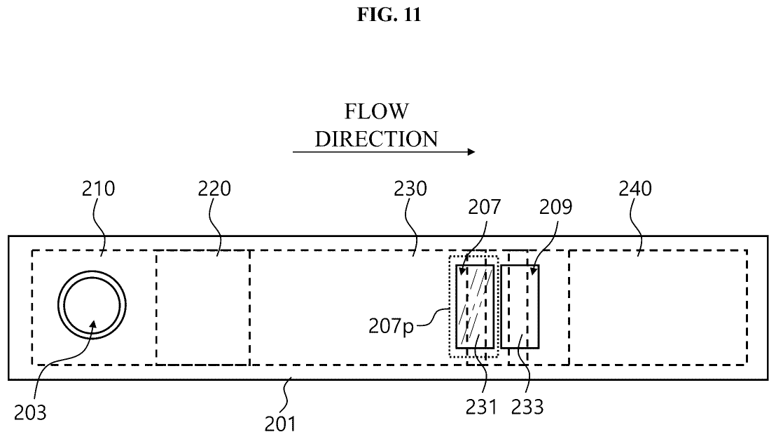

In this regard, FIG. 11 is an exemplary diagram of the arrangement of the light blocking film of the test strip according to one aspect of the present disclosure.

As illustrated in FIG. 11, according to one aspect of the present disclosure, the housing 201 may include the first opening 203 disposed above the absorption pad 240, a test area opening 207 exposing at least a portion of the test area 231 to the outside, and a control area opening 209 exposing at least a portion of the control area 233 to the outside. That is, the opening for the test area 231 and the opening for the control area 233 may be provided separately. According to one aspect, the test strip 200 may further include a light blocking film 207p having transparency equal to or less than a predetermined third transparency and covering the test area opening 207. That is, the influence of light on the metal nano probe or Raman indicator existing in the test area 231 can be minimized based on the light blocking film 207p having a transparency lower than a predetermined light transparency.

Here, it should be understood that the fact that the opening 207 for the test area 231 and the opening 209 for the control area 233 are provided includes not only forming two physically separated openings, but also separating the area corresponding to the test area 231 and the part corresponding to the control area 233 by providing the light blocking film 207p in one opening.

Meanwhile, according to one aspect, the light blocking film 207p is configured to be removable, discoloration of the control area may be exposed based on the control area opening 209, and the light blocking film 207p may be removed prior to the Raman spectroscopic analysis. That is, for example, the light transparency of the light blocking film 207p may be so low that it is not possible to check whether or not the color development of the test area 231 covered by the light-blocking film 207p is detected. Nevertheless, since it is possible to secure the view of the control area 233 through the opening 209, an experimenter on the test strip according to one aspect of the present disclosure can confirm that color development proceeds in the control area 233 after the body fluid sample and/or the washing liquid are introduced. That is, the experimenter can check the reaction situation of the analyte to the test strip based on the control area 233. When the Raman spectroscopic analysis is reached through a series of procedures, the experimenter may perform the Raman spectroscopic analysis by removing the detachable light blocking film 207p covering the test area 231.

### Cutting Unit

Meanwhile, as described above in relation to the method of measuring the concentration of the analyte in the bodily fluid sample according to one aspect of the present disclosure, according to one aspect, the test strip 200 may further include the cutting unit for cutting at least a portion of the absorption pad 240 after the washing liquid is absorbed by the absorption pad 240. Accordingly, it is possible to prevent the washing liquid absorbed by the absorption pad 240 from flowing backward toward the detection pad 230.

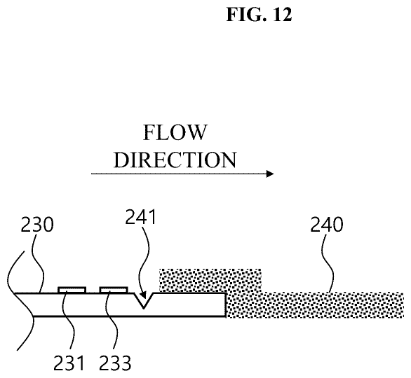

FIG. 12 is an exemplary diagram of a trench for cutting the absorption pad of the test strip according to one aspect of the present disclosure. As illustrated in FIG. 12, according to one aspect of the present disclosure, the cutting unit may be a trench 241 formed in the detection pad 230 or the absorption pad 240 in the width direction of the test strip. Although the trench 241 is illustrated as being provided in the detection pad 230 in FIG. 12, the present disclosure is not limited thereto, the trench 241 may be provided in the absorption pad 240 so that a portion of the absorption pad 240 is cut.

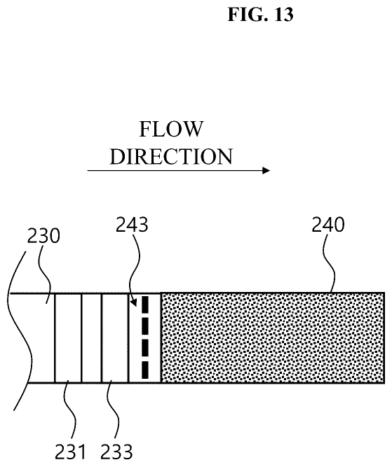

FIG. 13 is an exemplary diagram of a cutting line for cutting the absorption pad of the test strip according to one aspect of the present disclosure. As illustrated in FIG. 13, the cutting unit may be a cutting line 243 including cutting portions and coupling portions alternately formed in the detection pad 230 or the absorption pad 240 in the width direction of the test strip. Although the cutting line 243 is illustrated as being provided in the detection pad 230 in FIG. 13, the present disclosure is not limited thereto, the cutting line 243 may be provided in the absorption pad 240 so that a portion of the absorption pad 240 is cut.

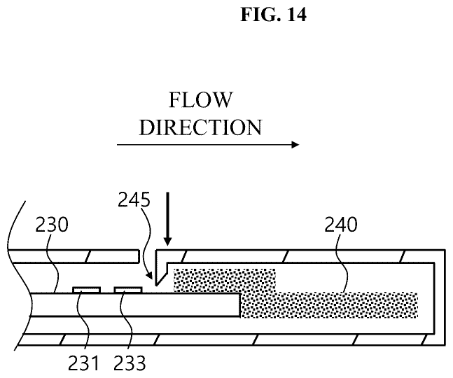

FIG. 14 is an exemplary diagram of a cutting tool for cutting the absorption pad of the test strip according to one aspect of the present disclosure. As illustrated in FIG. 14, the cutting unit may be a cutting tool 245 coupled to an operation unit outside the housing 201 and configured to cut the detection pad 230 or the absorption pad 240 by controlling the operation unit. The operation unit may adopt any structure in which the cutting tool 245 can cut the detection pad 230 or the absorption pad 240 by pressing, as illustrated by a downward arrow in FIG. 14. Although the cutting tool 245 is illustrated as cutting the detection pad 230 in FIG. 14, the present disclosure is not limited thereto, and the cutting tool 245 may be provided on an upper end of the absorption pad 240 to cut a portion of the absorption pad 240.

As described above, the test strip 200 according to the embodiments of the present disclosure may include at least one of cutting units according to various implementation methods. However, it should be understood that the technical spirit of the present disclosure is not limited thereto, and various cutting structures or tools implemented to cut the absorption pad 240 are included in the technical scope of the present disclosure.

### Blocking Unit

According to one aspect of the present disclosure, a blocking unit may be further provided, which is fixed after the absorption of the washing liquid by the absorption pad 240 to prevent the washing liquid from flowing backward from the absorption pad 240 to the detection pad 230. Accordingly, it is possible to prevent the washing liquid absorbed by the absorption pad 240 from flowing backward toward the detection pad 230.

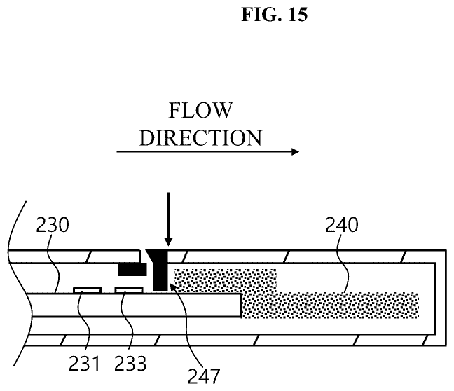

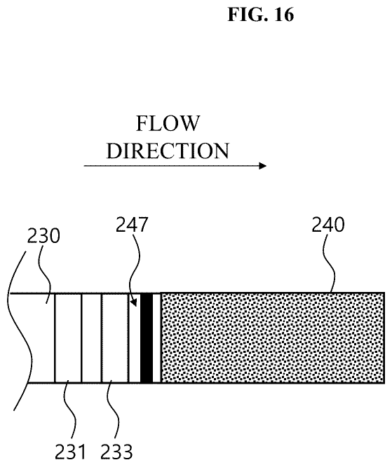

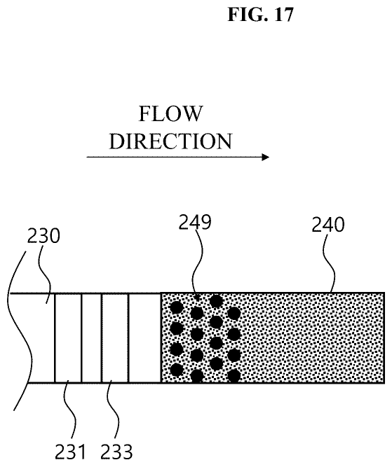

FIG. 15 is a side view of a pressurizing bulkhead for preventing backflow of the washing liquid in the test strip according to one aspect of the present disclosure, and FIG. 16 is a plan view of the pressurizing bulkhead for preventing backflow of washing liquid in the test strip according to one aspect of the present disclosure. As illustrated in FIGS. 15 and 16, according to one aspect, the blocking unit may be a pressurizing bulkhead 247 which is formed in the width direction of the detection pad 230 or the absorption pad 240 and presses the detection pad 230 or the absorption pad 240. As illustrated in FIG. 16, the pressurizing bulkhead 247 is disposed to be long in the width direction of the test strip 200, and as illustrated in FIG. 15, for example, the pressurizing bulkhead 247 is lowered by the pressurizing from above to pressurize the detection pad 230 or the absorption pad 240, and thus, prevents the backflow of the washing liquid. In addition, when the pressurizing bulkhead 247 is lowered, as illustrated in FIG. 15, an inclined portion is lowered in contact with a protruding portion, and when the inclined portion is lowered to the end, an end of the inclined portion is caught and fixed to the protruding portion to prevent the pressurizing bulkhead 247 from being raised. In FIG. 17, the pressurizing bulkhead 247 is illustrated as pressurizing the detection pad 230, but the present disclosure is not limited thereto, and the pressurizing bulkhead 247 may be provided at a position corresponding to the absorption pad 240 to press the absorption pad 240.

FIG. 17 is a plan view of the pressurizing protrusion for preventing the backflow of the washing liquid in the test strip according to one aspect of the present disclosure. As illustrated in FIG. 17, according to one aspect, the blocking unit may be a plurality of pressurizing protrusions 249 which are disposed in at least a partial area of the detection pad 230 or the absorption pad 240 and pressurizes the detection pad 230 or the absorption pad 240. In FIG. 17, the pressurizing protrusion 249 is illustrated as pressurizing the absorption pad 240, but the present disclosure is not limited thereto, and for example, the pressurizing protrusion 249 may be provided at a position after the control area 233 of the detection pad 230 to pressurize the detection pad 230.

As described above, the test strip 200 according to the embodiments of the present disclosure may include at least one of blocking units according to various implementation methods. However, it should be understood that the technical idea of the present disclosure is not limited thereto, and various blocking structures or tools implemented to prevent backflow from the absorption pad 240 are included in the technical scope of the present disclosure.

### Metal Nano Probe

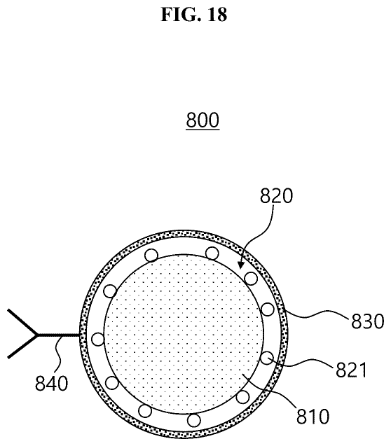

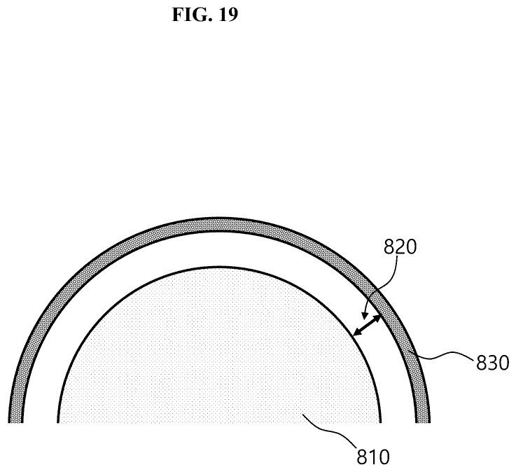

FIG. 18 illustrates an exemplary structure of the metal nano probe according to one aspect of the present disclosure, and FIG. 19 illustrates a hot spot formed in the metal nano probe of FIG. 18. A structure of an exemplary metal nano probe of the present disclosure will be described with reference to FIGS. 18 and 19.

According to one aspect of the present disclosure, a metal nano probe 800 may include a metal nano core 810 formed of a first metal, a Raman indicator layer 820 surrounding the metal nano core and having one or more Raman indicators 821, a metal shell 830 surrounding the Raman indicator layer and formed of a second metal, and a detection antibody 840 fixed to the metal shell and binding to an analyte.

That is, in order to form the metal nano probe 800 according to one aspect of the present disclosure, the metal nano core 810 is first formed of the first metal. According to one aspect, the metal nano core 810 may be formed of gold (Au). Silver (Ag) may be more advantageous in terms of surface-enhanced Raman scattering, but the metal nano core 810 may be formed of gold (Au) in consideration of the high initial molding difficulty due to the effect of zeta potential.

After that, at least one Raman indicator 821 may be immobilized on the outer surface of the metal nano core 810. Here, as the Raman indicator, any substance that generates Raman scattering, such as MBA or MGITC, can be used. When the Raman indicator is immobilized, a metal shell 830, which is a metal thin film, may be formed on the Raman indicator. According to one aspect, the metal shell may be formed of silver (Ag), which is more advantageous for the surface-enhanced Raman scattering.

The Raman indicator layer 820 including Raman indicators may be formed by fixing the plurality of Raman indicators to the core and forming a shell. Here, as illustrated in FIG. 19, the Raman indicator layer 820 forms a micro-space between the metal nano core 810 and the metal shell 830. That is, according to one aspect, the metal nano core 810 and the metal shell 830 may be spaced apart from each other by at least one Raman indicator to form the hot spot for generating the surface-enhanced Raman scattering in the Raman indicator layer 820.

The detection antibody 840 capable of binding to an analyte may be immobilized on the metal shell 830 to form the metal nano probe 800 according to one aspect of the present disclosure.

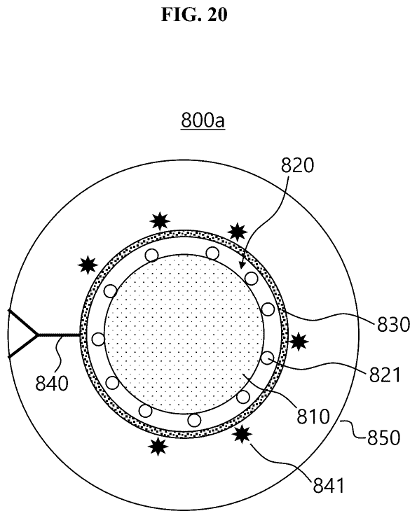

FIG. 20 illustrates an exemplary structure of a metal nano probe according to another aspect of the present disclosure. As illustrated in FIG. 20, according to one aspect of the present disclosure, a metal nano probe 800a may further includes at least one additional Raman indicator 841 disposed on an outer surface of the metal shell 830. Here, according to one aspect, the Raman indicator 821 and the additional Raman indicator 841 may be different from each other. That is, for example, one of the Raman indicator 821 and the additional Raman indicator 841 may be MBA, and the other may be MGITC. However, the present disclosure is not limited to this, and any one of various Raman indicators can be adopted as the Raman indicator 821 and the additional Raman indicator 841, respectively.

### Spectroscopic Analysis Method and System

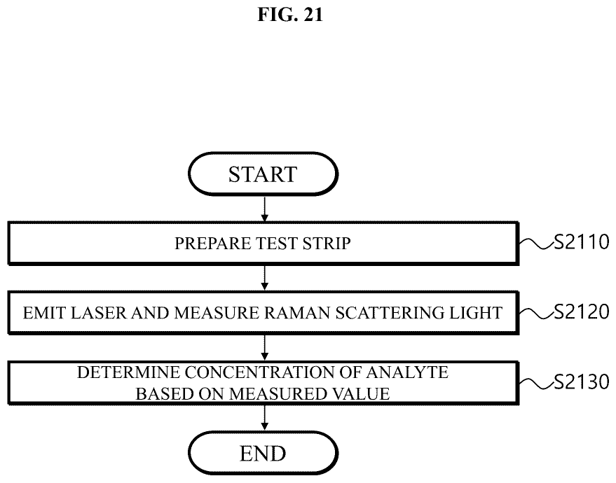

FIG. 21 is a schematic flowchart of a Raman spectroscopic analysis method of the test strip for measuring the concentration of the analyte in the bodily fluid sample according to one embodiment of the present disclosure. According to one aspect of the present disclosure, the concentration of the analyte in bodily fluid sample may be measured by performing the Raman spectroscopy on at least a portion of the test area and/or the control area of the test strip.

As illustrated in FIG. 21, according to one embodiment of the present disclosure, there is provided a Raman spectroscopic analysis method for a test strip for measuring a concentration of an analyte in a bodily fluid sample, the method including a step (S2110) of preparing a test strip in which an analyte is fixed in a spectroscopic analysis target area, a step (S2120) of irradiating the spectroscopic analysis target area with laser and measuring Raman scattering light from the spectroscopic analysis target area, and a step (S2130) of determining the concentration of the analyte based on a measured value of the Raman scattering light.

More specifically, according to the Raman spectroscopic analysis method of a test strip for measuring the concentration of analyte in a bodily fluid sample according to one embodiment of the present disclosure, first, the test strip in which the analyte is fixed in a spectroscopic analysis target area may be prepared (S2110).

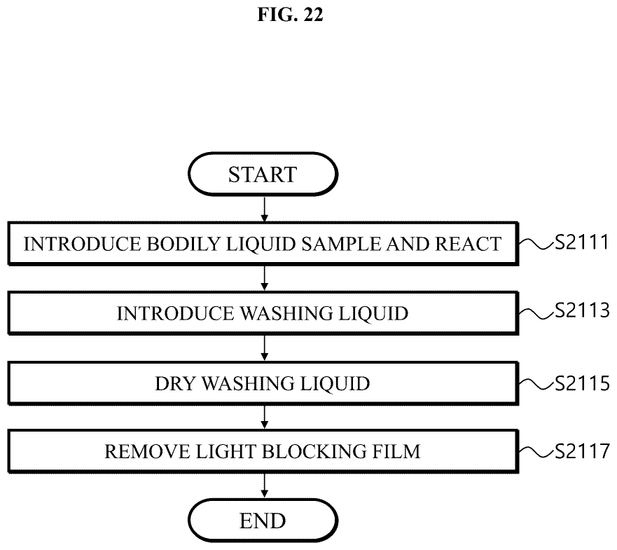

FIG. 22 is a detailed flowchart of the test strip preparation step of FIG. 21. As illustrated in FIG. 22, according to one aspect, first, the step (S2110) of preparing the test strip may be initiated by reacting by introducing the bodily fluid sample containing the analyte into the test strip for the lateral flow assay (LFA) (S2111). Thereafter, the washing liquid is introduced into the test strip after the predetermined first time interval has elapsed from the introduction of the bodily fluid sample (S2113). In the step (S2110) of preparing the test strip, at least some of the procedures described in relation to the method for measuring the concentration of the analyte in the bodily fluid sample according to one aspect of the present disclosure may be equally applied. For example, according to one aspect, the test area may be dried for a predetermined third time interval after the washing liquid is introduced (S2115). Moreover, according to one aspect, the step (S2117) of removing the light blocking film attached to the opening corresponding to the test area of the test strip housing may be performed.

Referring back to FIG. 22, the spectroscopic device may be used to irradiate the spectroscopic analysis target area with laser and measure the Raman scattering light from the spectroscopic analysis target area (S2120).

Here, the area which is target of the spectroscopic analysis may be set to at least a part of at least one of the test area 2231 and/or the control area 2133 to improve analysis accuracy.

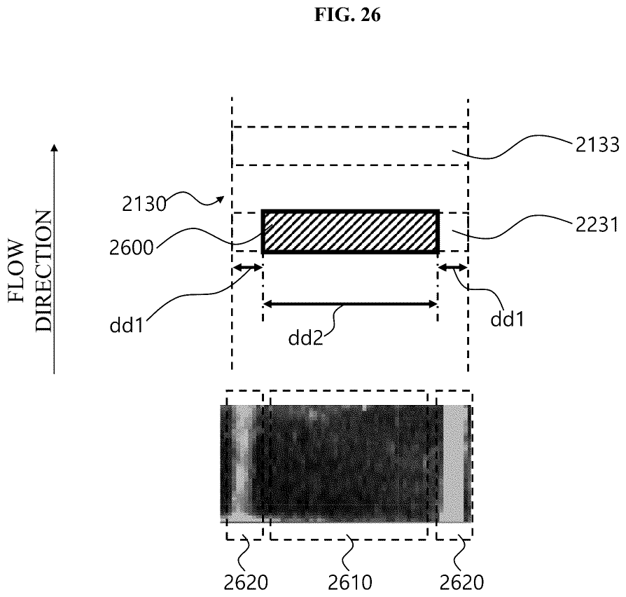

FIG. 26 is an exemplary diagram of the target area for the Raman spectroscopic analysis according to one aspect of the present disclosure.

As illustrated in FIG. 26, according to one aspect, a spectroscopic analysis target area 2600 may be configured to have a width that is reduced from both side surfaces by a first predetermined length dd1 or more than a width of a test area 2231. More specifically, according to one aspect, the first length dd1 may be determined such that a side area 2620 of the test area 2231 in the width direction is excluded from the spectroscopic analysis target area 2600.

The side area 2620 in the width direction may be an area in which the density of the metal nano probes remaining after transport of the bodily fluid sample and the washing liquid is different from the density of the metal nano probes in a center area 2610 in the width direction by a predetermined threshold error or more. In other words, the side area 2620 in the width direction may be an area in which the density of the fixed analyte differs from the density of the analyte in the center area 2610 in the width direction by a predetermined threshold error or more.

As illustrated in FIG. 26, it can be seen that excessively strong Raman scattering light is detected in both side areas 2620 of the test area in the width direction. This characteristic is due to the nature of a lateral flow assay (LFA), as the bodily fluid sample and/or washing liquid transport along the flow direction while carrying the nano-metal probe including the Raman indicator. Accordingly, it is assumed that more nanometallic probes are captured or retained in both side areas of the flow direction. According to one aspect of the present disclosure, the side areas 2620 in the width direction in which an excessively high intensity of Raman scattering light is detected compared to the center area in the width direction may be excluded from the target area of Raman spectroscopic analysis.

However, according to one aspect, the spectroscopic analysis target area 2600 may be configured to have a width capable of securing a predetermined number or more of Raman spectroscopic analysis points. For example, as illustrated in FIG. 26, according to one aspect of the present disclosure, spectroscopic analysis of the test area 2231 is performed on a plurality of Raman spectroscopic analysis points in the width direction and the length direction. Each pixel of FIG. 26 may represent an analysis result for one spectroscopic analysis point. The size of the point may vary according to the setting, but in order to secure a Raman spectroscopic analysis mapping result with a desired resolution, the desired number of spectroscopic analysis points according to the set size should be included in the width direction. When the width of the spectroscopic analysis target area 2600 is too small, only a limited number of spectroscopic analysis points can be arranged in the width direction. Therefore, the width of the spectroscopic analysis target area 2600 may not be reduced as compared with the length at which the desired number of spectroscopic analysis points can be arranged.

According to one aspect, the step (S2120) of measuring of the Raman scattering light may be performed before a predetermined threshold time interval has elapsed from the completion of the preparation of the test strip. As described above, since the light exposure to the Raman indicator can reduce intensity according to the Raman spectroscopic analysis, the Raman scattering light measurement step may be performed before a predetermined threshold time interval has elapsed.

Meanwhile, according to one aspect of the present disclosure, the target of the spectroscopic analysis is the metal nano probe which includes the Raman indicator and the metal nanoparticles to generate the nano surface-enhanced Raman scattering. However, when the laser emitted for the spectroscopic analysis on the metal nanoparticles such as gold (Au) contains excessively strong energy, combustion of the metal nano probe occurs, and there is problem that the intensity of the spectroscopic analysis decreases. According to one aspect of the present disclosure, a laser having a beam spot size exceeding a first size generating the combustion of the metal nano probe to which an analyte binds may be irradiated. Such a beam spot size may be performed by adjusting magnification, for example. When the beam spot size is reduced by adjusting the magnification when the laser containing the same energy is emitted, the energy is concentrated in a small area, and the combustion of the metal nano probe may occur. Accordingly, the beam spot size according to one aspect of the present disclosure may be configured to have a size that exceeds the size at which energy is concentrated to generate the combustion of the metal nano probe.

Meanwhile, when the beam spot size is too large, the analysis result image for the Raman spectroscopic analysis mapping has too low resolution. When the beam spot size increases within the limited spectroscopic analysis target area, the number of Raman spectroscopic analysis points that can be secured in the longitudinal direction and/or width direction decreases, and thus, a low-resolution spectroscopic analysis result occurs. According to one aspect, a laser having a beam spot size of a second size or less capable of securing a predetermined number or more of Raman spectroscopic analysis points within a spectroscopic analysis target area may be emitted.

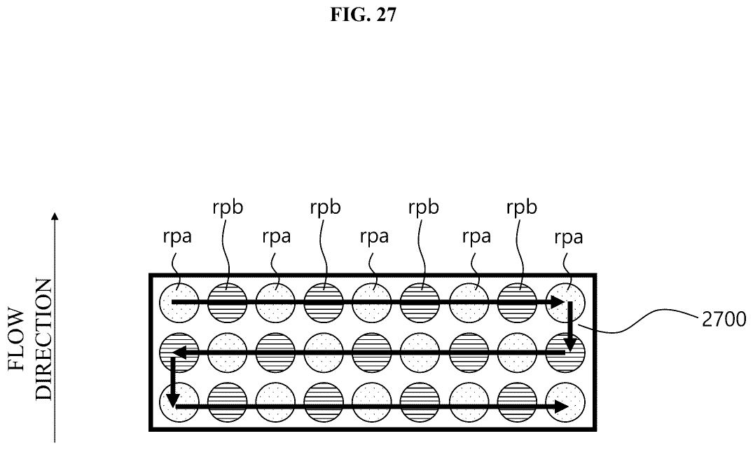

FIG. 27 illustrates the Raman spectroscopic analysis points and a moving path of the spectroscopic device according to one aspect of the present disclosure. As exemplarily illustrated in FIG. 27, a plurality of Raman spectroscopic analysis points (rpa, rpb) may be included in the spectroscopic analysis target area according to one aspect of the present disclosure.

According to one aspect, scattering light for a plurality of Raman spectroscopic analysis points within the analysis target area may be measured using a stage apparatus configured to horizontally move the spectroscopic device in any one of the longitudinal direction and the width direction within the analysis target area. The measurement result of the scattering light for each of the Raman spectroscopic analysis points may be expressed as a pixel value, and for example, a Raman spectroscopic analysis result image as illustrated in FIG. 26 may be derived.

However, as illustrated in FIG. 27, when the spectroscopic analysis is performed while moving along a moving path 2700 for a plurality of neighboring Raman spectroscopic analysis points, energy of the laser emitted to one spectroscopic analysis point is partially transmitted to the surrounding spectroscopic analysis points. As a result, energy higher than the set energy is transmitted to the spectroscopic analysis point, causing combustion and reducing the intensity (Intensity) which is the analysis result.

In order to solve this problem, according to one aspect, the spectroscopic device may be configured to stop the irradiation of the laser while moving from a first spectroscopic analysis point to a second spectroscopic analysis point among a plurality of Raman spectroscopic analysis points. When the laser is continuously emitted while moving between spectroscopic analysis points, for example, energy from the laser may be partially transmitted to the second spectroscopic analysis point even while moving from the first spectroscopic analysis point to the second spectroscopic analysis point. Therefore, even when the spectroscopic device reaches the second spectroscopic analysis point and emits the laser for spectroscopic analysis for a set time, as a result, as a result, the second spectroscopic analysis point receives more energy than expected, and the combustion of the metal nano probe may occur. Therefore, according to one aspect of the present disclosure, the spectroscopic device may stop the irradiation of the laser during the movement between the analysis points.

According to another aspect, in the analysis of a plurality of spectroscopic analysis points, the neighboring analysis points are not subject to continuous analysis, and a certain separation distance is guaranteed between analysis points that are subject to continuous spectroscopic analysis, thereby preventing unwanted energy transmission.

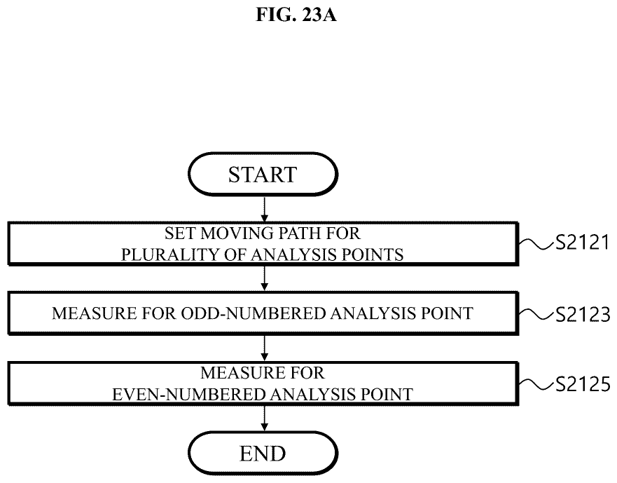

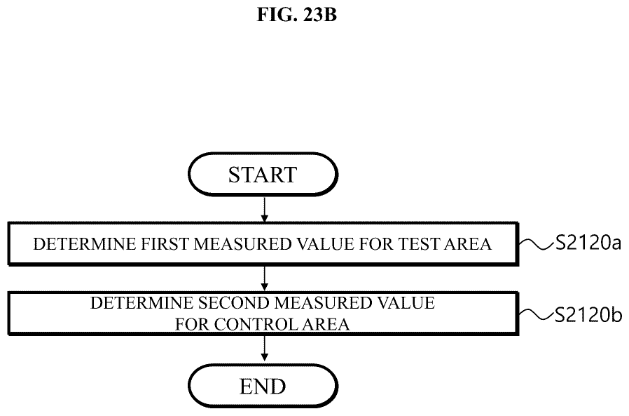

FIG. 23A is a detailed flowchart of the laser irradiation and the Raman scattering light measurement steps of FIG. 21. The spectroscopic analysis procedure with the separation distance will be described with reference to FIGS. 23A and 27. As illustrated in FIG. 23A, first, a moving path 2700 of the spectroscopic device for the plurality of Raman spectroscopic analysis points (rpa, rpb) may be set (S2121).

Within the moving path 2700, for example, the Raman scattering light is first measured for at least some of the odd-numbered Raman spectroscopic analysis points (rpa) (S2123), and then within the moving path 2700, the Raman scattering light may be measured for at least some of the even-numbered Raman spectroscopic analysis points (rpb) (S2125). Therefore, even when the irradiation of the laser to the analysis point (rpa) causes the energy transfer to some analysis points (rpb), when the spectroscopic analysis of the analysis point (rpa) is completed, the laser is not emitted to the analysis point (rpb) immediately, and the analysis of the next analysis point (rpa) is performed. Accordingly, it is possible to prevent the occurrence of combustion of the metal nano probe for the analysis point (rpb), thereby preventing the decrease in the intensity of the analysis result.

Referring back to FIG. 21, the concentration of the analyte is determined (S2130) based on the measured value of the Raman scattering light using an arithmetic device.

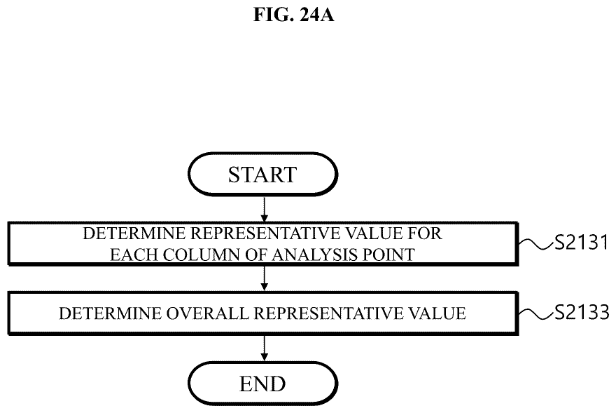

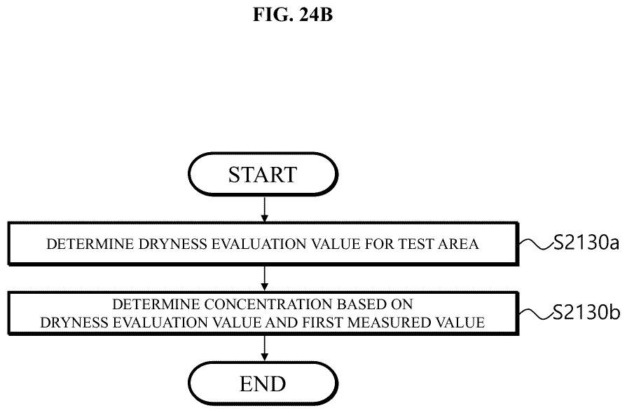

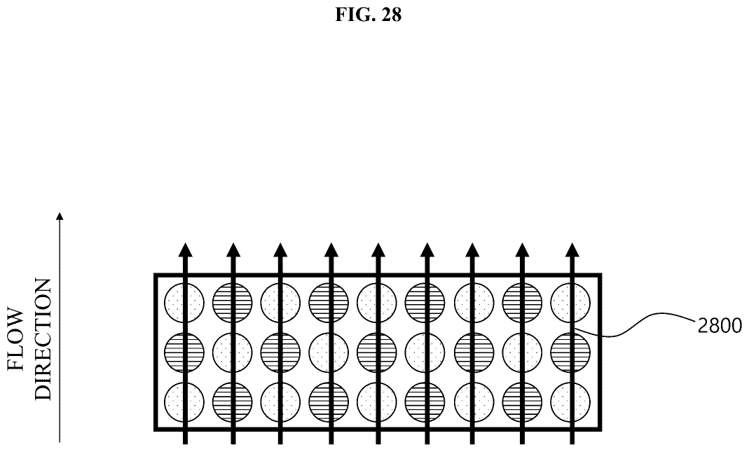

FIG. 24A is a detailed flowchart of the step of determining the concentration of the analyte based on the measured value of FIG. 21, and FIG. 28 is an exemplary diagram of determining a representative value based on the spectroscopic analysis measured value according to one aspect of the present disclosure. As illustrated in FIG. 24A, according to one aspect, in the step of determining the concentration of the analyte is, first, for columns 2800 of the Raman spectroscopic analysis points arranged in the longitudinal direction of the spectroscopic analysis target area, a representative value of the measured value for each column 2800 may be determined (S2131). In the test area of the test strip for the lateral flow analysis, the intensity of the signal according to the Raman spectroscopic analysis tends to change along the length direction. For example, it may have a tendency to increase in the first half of the test area along the length direction and gradually decrease after passing the highest point. In consideration of this point, according to one aspect of the present disclosure, for the measured values of the plurality of spectroscopic analysis points, first, a representative value of each measured value for each column in the longitudinal direction may be determined. Here, the representative value may be, for example, an average value or a median value, but is not limited thereto, and an arbitrary calculation method may be applied to represent measured values of points of each column.

Referring back to FIG. 24A, an overall representative value for the spectroscopic analysis target area may be determined based on representative values for each column (S2133). Therefore, the spectroscopic analysis value for the spectroscopic analysis target area of the test strip may be expressed as one value.

Meanwhile, according to one aspect of the present disclosure, the concentration of the analyte may be determined by further reflecting not only the spectroscopic analysis measured value of the test area but also the spectroscopic analysis measured value of the control area.

In this regard, FIG. 23B is a second detailed flow chart of the laser irradiation and the step of the measuring of the Raman scattering light in FIG. 21, and FIG. 24B is a second detailed flow chart of the step of determining the measured value-based concentration of analyte in FIG. 21.

According to one aspect of the present disclosure, the step of measuring the Raman scattering light (S2120) includes a step (S2120a) of determining the first measured value for the test area and a step (S2120b) of determining a second measured value for the control area as illustrated in FIG. 23B. Here, in the step of determining the concentration of the analyte (step 2130), the concentration of the analyte may be determined based on the first measured value and the second measured value.

In more detail, but not exclusively, as illustrated in FIG. 24B, the step (S2130) of determining the concentration of the analyte may include a step (S2130a) of determining the dryness evaluation value for the test area based on at least one of the first measured value and the second measured value, and a step (S2130b) of determining the concentration of the analyte according to the dryness evaluation value and the first measured value (step 2130b).

In other words, according to one aspect of the present disclosure, in determining the concentration of the analyte, not only the spectroscopic analysis result of the test area but also the spectroscopic analysis result of the control area may be further considered. As described above, when the test area and/or the control area are wet by the bodily fluid sample and/or the washing liquid, the intensity of the signal according to spectroscopic analysis may vary accordingly. Therefore, the dryness evaluation value indicating how dry the test area may be determined by comparing the first measured value of the test area according to the spectroscopic analysis and the second measured value of the control area according to the spectroscopic analysis with each other, and the concentration of the analyte according to the first measured value may be determined by reflecting the dryness evaluation value. For example, when the dryness degree is determined as the second value indicating less dryness than the first value, for the signal intensity for the same test area, a higher concentration may be determined when the drying degree is the second value than when the first value.

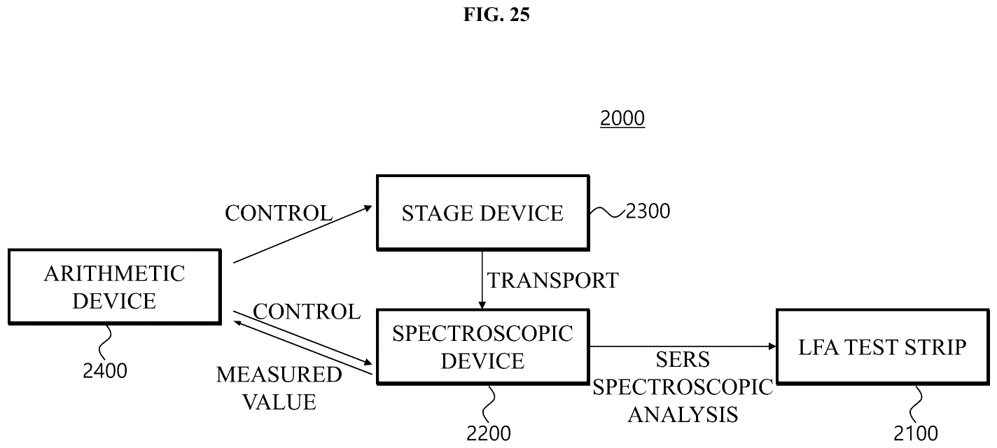

FIG. 25 illustrates a schematic configuration of the Raman spectroscopic analysis system according to one embodiment of the present disclosure. As illustrated in FIG. 25, a Raman spectroscopic analysis system 2000 according to one embodiment of the present disclosure may include an LFA test strip 2100, a spectroscopic device 2200, a stage device 2300, and an arithmetic device 2400.

The test strip 2100 may be configured such that the analyte is fixed to the spectroscopic analysis target area in order to measure the concentration of the analyte in the bodily fluid sample.

The spectroscopic device 2200 may be configured to irradiate the spectroscopic analysis target area of the test strip with laser and measure the Raman scattering light from the spectroscopic analysis target area.

The stage device 2300 may be configured to horizontally move the spectroscopic device in any one of the longitudinal direction and the width direction within the analysis target area.

The arithmetic device 2400 may be configured to determine the concentration of the analyte based on a measured value of the Raman scattering light measured by the spectroscopic device. Moreover, the arithmetic device may be configured to perform control of the stage device and the spectroscopic device. For example, the arithmetic device may be a computing device having a processor and memory.

The spectroscopic analysis method according to one aspect of the present disclosure described above may be performed using the above spectroscopic analysis system or configuration thereof.

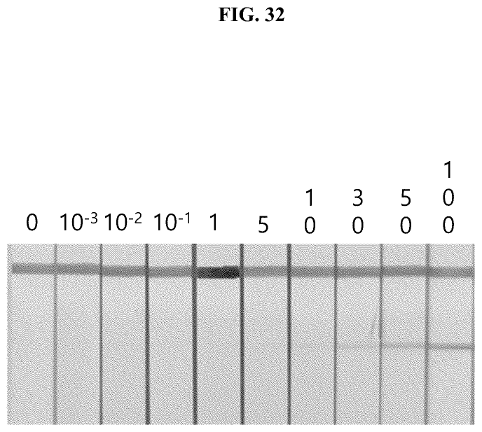

FIG. 32 illustrates response results of the test area at each concentration. As illustrated in FIG. 32, color development of the test area can be visually confirmed for an analyte of a predetermined concentration or more depending on the concentration of the analyte.

FIG. 33 illustrates the results of the Raman spectroscopic analysis at each concentration. As illustrated in FIG. 33, it was confirmed that the higher the concentration of the analyte, the greater the intensity of the signal according to Raman spectroscopic analysis. In addition, it was also confirmed that excessively high strength generated in the side area in the width direction of the test area compared to the central area.

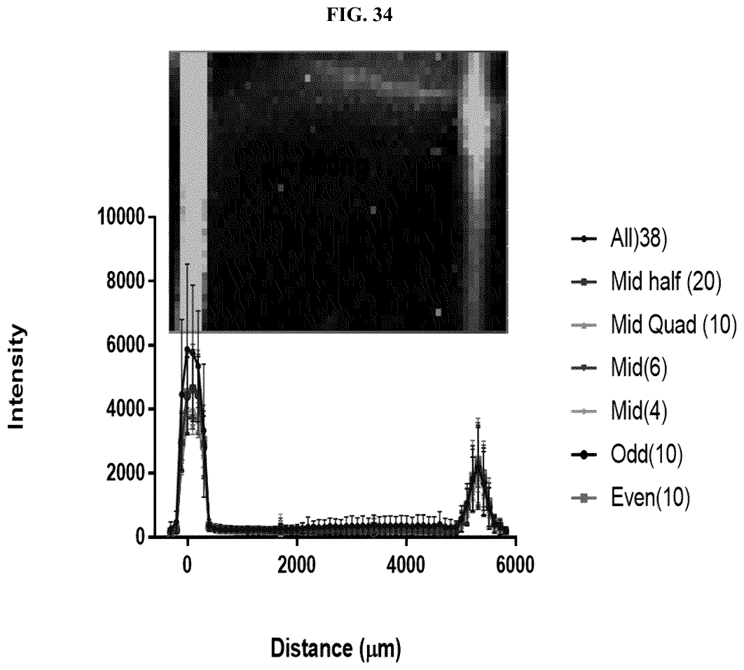

FIG. 34 illustrates the change of the Raman spectroscopic analysis measured value from one side of the test area to the other side. As described above, it is possible to determine a representative value for each of a plurality of columns of the spectroscopic analysis target area, and the measured value of each column may be displayed according to various criteria, such as the values of all points of the corresponding column, even-numbered points, odd-numbered points, or a predetermined number of points in the center. As illustrated in FIG. 34, it can be confirmed that each of the side areas in the width direction has a higher error than the center area for these various criteria.

FIG. 35 illustrates the difference between the Raman spectroscopic analysis results for the test area before and after drying, and FIG. 36 illustrates the analysis result of the Raman spectroscopic analysis results of FIG. 35.

As illustrated in FIGS. 35 and 36, it was confirmed that intensity appeared high in an average (3520a) or total (3520s) of each column after 15 minutes (3520) compared to an average (3510a) or total (3510s) of each column in a case (3510) where the test area of the test strip was completely wet.

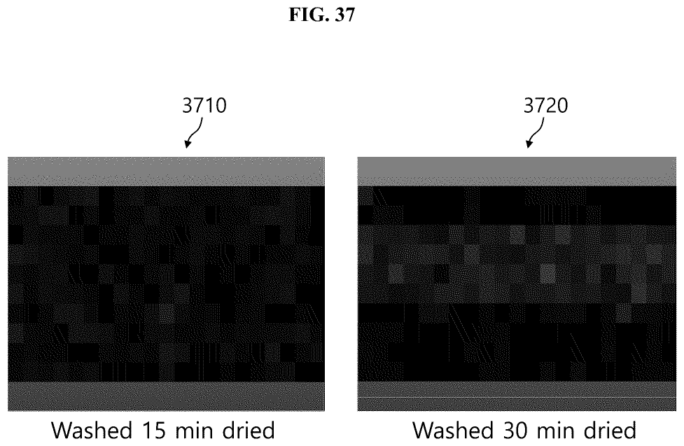

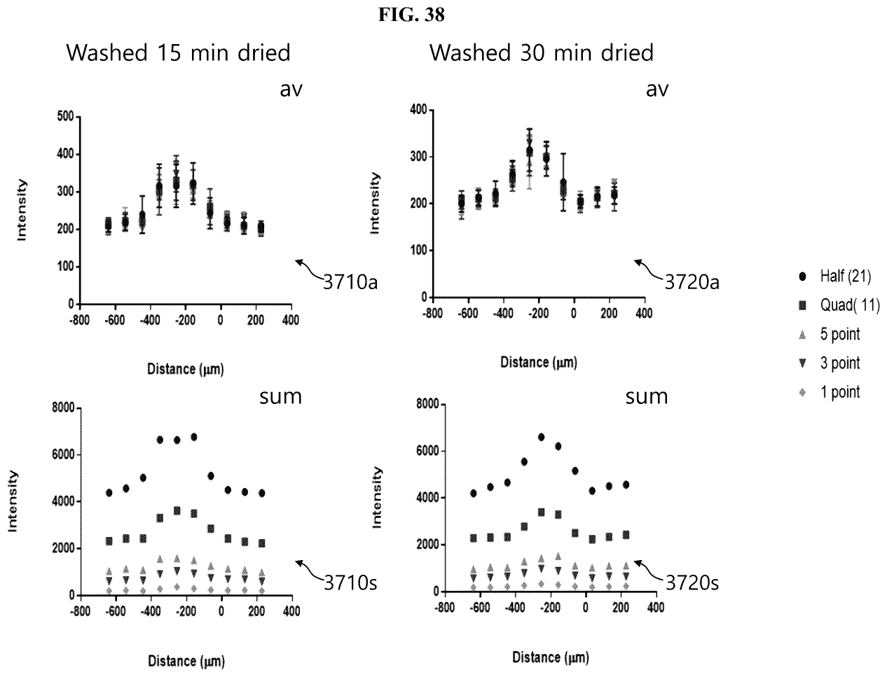

FIG. 37 illustrates differences in Raman spectroscopic analysis results for the test area over time after washing, and FIG. 38 illustrates analysis results of the Raman spectroscopic analysis results of FIG. 37.

As illustrated in FIGS. 37 and 38, it was confirmed that intensity appeared high in an average (3720a) or total (3720s) of each column in a case (3720) after the drying was performed for 30 minutes compared to an average (3710a) or total (3710s) of each column in a case (3710) where the washing was performed and the drying was performed for 15 minutes.

Although the above has been described with reference to the drawings and examples, it does not mean that the scope of protection of the present disclosure is limited by the drawings or examples, and those skilled in the art will understand the scope of the present disclosure described in the claims below. It will be understood that various modifications and changes can be made to the present disclosure within the scope not departing from the spirit and area.

Specifically, operations by, for example, an arithmetic device among the described features may be executed within a digital electronic circuit, or computer hardware, firmware, or combinations thereof. Features may be implemented in a computer program product embodied within storage, that is, in a machine-readable storage device, for execution by a programmable processor. And features can be performed by a programmable processor executing a program of instructions to perform the functions of the described embodiments by operating on input data and generating output. The described features include at least one programmable processor, at least one input device, and at least one output device coupled to receive data and instructions from and to transmit data and instructions to the data storage system. It can be executed within one or more computer programs that can be executed on a programmable system including. A computer program includes a set of instructions that can be used directly or indirectly within a computer to perform a particular action for a given result. A computer program is written in any programming language, including compiled or interpreted languages, and contained as modules, components, subroutines, or other units suitable for use in other computer environments, or as stand-alone programs.

Suitable processors for execution of a program of instructions include, for example, both general and special purpose microprocessors, and either a single processor or multiple processors in a computer of another kind. Moreover, storage devices suitable for embodying computer program instructions and data embodying the described features include, for example, semiconductor memory devices such as EPROM, EEPROM, and flash memory devices, magnetic memory devices such as internal hard disks and removable disks, and all forms of non-volatile memory including magneto-optical disks and CD-ROM and DVD-ROM disks. The processor and memory may be integrated within or added by application-specific integrated circuits (ASICs).

Although the present disclosure described above has been described based on a series of functional blocks, it is not limited by the above-described embodiments and it will be clear to those skilled in the art that various substitutions, modifications, and changes are possible within the scope of the technical idea of the present invention.

Combinations of the above-described embodiments are not limited to the above-described embodiments, and various types of combinations may be provided as well as the above-described embodiments according to implementation and/or needs.

In the foregoing embodiments, the methods are described on the basis of a flow chart as a series of steps or blocks, but the present disclosure is not limited to the order of steps, and some steps may occur in a different order or concurrently with other steps as described above. In addition, those skilled in the art will understand that the steps illustrated in the flow chart are not exclusive, that other steps may be included, or that one or more steps of the flow chart may be deleted without affecting the scope of the present disclosure.

The foregoing embodiment includes examples of various aspects. It is not possible to describe all possible combinations to represent the various aspects, but those skilled in the art will recognize that other combinations are possible. Accordingly, it is intended that the present disclosure cover all other substitutions, modifications and variations falling within the scope of the following claims.

What is claimed is:

1. A method of measuring a concentration of an analyte in a bodily fluid sample, the method comprising:
introducing the bodily fluid sample containing the analyte into a test strip for a lateral flow assay (LFA) by a first volume to react the analyte with a metal nano probe for surface-enhanced Raman scattering (SERS);
introducing a washing liquid into the test strip by a second volume after a first predetermined time interval has elapsed from the introduction of the bodily fluid sample; and
performing a surface-enhanced Raman scattering (SERS)-based spectroscopic analysis on at least one of a test area and a control area of the test strip using a spectroscopic device.

2. The method of claim 1, wherein the introducing of the bodily fluid sample by the first volume to react includes
loading the bodily fluid sample by a sample pad of the test strip,
binding the metal nano probe including a Raman indicator and a detection antibody included in a conjugate pad of the test strip and the analyte included in the bodily fluid sample, and
transporting the bodily fluid sample by an absorption pad provided on a side surface opposite to a side surface of the conjugate pad of the detection pad of the test strip, capturing a complex of the metal nano probe and the analyte by a capture antibody fixed to the test area of the detection pad, and capturing the metal nano probe by the control area of the detection pad.

3. The method of claim 2, wherein the introducing of the washing liquid by the second volume includes transporting the washing liquid loaded on the sample pad via the conjugate pad and the detection pad by the absorption pad.

4. The method of claim 3, further comprising, after the introducing of the washing liquid, transporting complexes located at an area prior to the test area of the detection pad or the conjugate pad among complexes of the metal nano probe and the analyte to the test area by the washing liquid.

5. The method of claim 3, further comprising, after the introducing of the washing liquid, removing non-specific binding by substances other than the analyte to the capture antibody of the test area by the washing liquid.

6. The method of claim 3, wherein the second volume of the washing liquid is set to be equal to or greater than a first threshold volume which is a volume allowing complexes located at an area prior to the test area of the detection pad or the conjugate pad among complexes of the metal nano probe and the analyte to be transported to the test area.

7. The method of claim 6, wherein the second volume of the washing liquid is set to be smaller than a second threshold volume which is a volume allowing the complex of the metal nano probe and the analyte captured by the capture antibody of the test area to be lost.

8. The method of claim 7, wherein at least one of the first threshold volume and the second threshold volume of the washing liquid is determined based on at least one of
a first volume of the bodily fluid sample,
a viscosity of the bodily fluid sample,
a viscosity of the washing liquid,
a type of the analyte,
a type of the metal nano probe, or
an absorbent volume of the absorbent pad.

9. The method of claim 3, wherein the absorption pad has an absorbent volume equal to or greater than a sum of the first volume and the second volume.

10. The method of claim 3, further comprising cutting at least a portion of the absorbent pad of the test strip after a second predetermined time interval has elapsed from the introduction of the washing liquid,
wherein the SERS-based spectroscopic analysis is performed after the cutting of a portion of the absorption pad.

11. The method of claim 3, further comprising drying the test area for a predetermined third time interval after the washing liquid is introduced,
wherein the SERS-based spectroscopic analysis is performed after the drying of the test area.

12. The method of claim 2, wherein the metal nano probe includes
a metal nano core formed of a first metal,
a Raman indicator layer surrounding the metal nano core and having one or more Raman indicators,
a metal shell surrounding the Raman indicator layer and formed of a second metal, and
a detection antibody fixed to the metal shell and binding to the analyte.

13. The method of claim 12, wherein the metal nano core and the metal shell are spaced apart from each other by the at least one Raman indicator to form a hot spot for generating the surface-enhanced Raman scattering in the Raman indicator layer.

14. The method of claim 12, wherein the metal nano core is formed of gold (Au), and
the metal shell is formed of silver (Ag).

15. The method of claim 12, wherein the metal nano probe further includes at least one additional Raman indicator disposed on an outer surface of the metal shell.

16. The method of claim 15, wherein the Raman indicator and the additional Raman indicator are different from each other.

17. The method of claim 15, wherein one of the Raman indicator and the additional Raman indicator is MBA, and
the other of the Raman indicator and the additional Raman indicator is MGITC.

18. The method of claim 11, wherein the performing of the SERS-based spectroscopic analysis includes
determining a first measured value of the test area based on spectroscopic analysis,
determining a second measured value of the control area based on spectral analysis,
determining a dryness evaluation value of the test area based on at least one of the first measured value and the second measured value, and
determining the concentration of the analyte according to the dryness evaluation value and the first measured value.

19. A system of measuring a concentration of an analyte in a bodily fluid sample, the system comprising:
a spectroscopic device and a test strip for a lateral flow assay,
wherein the test strip receives the bodily fluid sample including an analyte by a first volume, reacts the analyte with a metal nano probe for surface-enhanced Raman scattering, and receives a washing liquid by a second volume after a first predetermined time interval from the introduction of the bodily fluid sample, and
the spectroscopic device performs a surface-enhanced Raman scattering-based spectroscopic analysis on at least one of a test area and a control area of the test strip.
